(19) United States
(12) Patent Application Publication
(10) Pub. No.: US 2013/0225603 A1
(43) Pub. Date: Aug. 29, 2013

(54) MDM2 INHIBITORS FOR TREATMENT OF OCULAR CONDITIONS

(71) Applicants: Sai Chavala, Durham, NC (US); Thomas Cramer Lee, Altadena, CA (US)

(72) Inventors: Sai Chavala, Durham, NC (US); Thomas Cramer Lee, Altadena, CA (US)

(73) Assignee: SERRATA LLC, DURHAM, NC (US)

(21) Appl. No.: 13/876,408

(22) PCT Filed: Sep. 26, 2011
(86) PCT No.: PCT/US11/53267

§ 371 Date: May 14, 2013

Related U.S. Application Data
(60) Provisional application No. 61/386,808, filed on Sep. 27, 2010.

(51) Int. Cl. A61K 31/496; A61K 45/06; A61K 9/00

(52) U.S. Cl. CPC A61K 31/496; A61K 9/0048; A61K 45/06
USPC 514/254.05

(57) ABSTRACT

Provided herein are pharmaceutical compositions for the treatment of various ocular diseases characterized by unwanted cellular proliferation. The pharmaceutical compositions may comprise one or more MDM2 inhibitors, and may further comprise one or more additional therapeutic agents. Also provided are methods of use of MDM2 inhibitors and/or formulations thereof for the treatment of ocular diseases characterized by unwanted cellular proliferation.

### FIELD OF THE INVENTION

The present application is directed to methods of use of MDM2 inhibitors. In particular, it relates to the use of MDM2 inhibitors to prevent and/or treat various diseases of the eye.

### BACKGROUND OF THE INVENTION

Abnormal retinal vascular proliferation is implicated in diseases such as age-related macular degeneration (ARMD), proliferative diabetic retinopathy, and retinopathy of prematurity. One common treatment for such proliferative diseases is the use of neutralizing antibodies to vascular endothelial growth factor-A (VEGF-A). However, not all patients exhibit the intended response to anti-VEGF therapy. This may be due to the fact that other cytokines may also contribute to retinal proliferation independent of VEGF. Therefore, using a targeted cytokine approach such as the current anti-VEGF therapy may not completely inhibit pathologic angiogenesis due to compensation of other untargeted cytokines.

Recent reports suggest that the p53 pathway may participate in the regulation of angiogenesis. The tumor suppressor protein, p53, is a major transcription factor that protects cells from malignant transformation and is mutated in many cancers. This protein is considered the master regulator of cell cycle arrest, senescence, and apoptosis. Various events, such as DNA damage by radiation or UV light and cellular stress, lead to post-translational modification of p53. Under non-stressed conditions, p53 is tightly controlled by one of its downstream targets, MDM2 (Murine Double Minute2), which targets p53 for ubiquitin mediated proteolysis, resulting in an auto-regulatory feedback loop. Inhibition of MDM2 leads to an accumulation of p53, which in turn results in cell cycle arrest or apoptosis.

Given the shortcomings of anti-VEGF therapy in certain cases, it would be advantageous to identify other therapeutic treatments for abnormal retinal vascular proliferation and angiogenesis, such as therapies that modulate the p53 pathway or otherwise elicit a biological response distinct from anti-VEGF therapy.

### BRIEF SUMMARY OF THE INVENTION

In one aspect of the present invention is provided a method of treating a disease or condition associated with unwanted cellular proliferation in the eye. In certain embodiments, the method comprises administering to the subject an effective amount of an MDM2 inhibitor. In some embodiments, the MDM2 inhibitor is a nutlin compound. In one specific embodiment, the nutlin compound is Nutlin-3.

In certain embodiments, the method relates to the treatment of ocular conditions including age-related macular degeneration, retinopathy of prematurity, diabetic retinopathy, proliferative vitreoretinopathy, ocular melanoma, ocular lymphoma, retinal vein occlusions, sickle cell retinopathy, choroidal hemangioma, choroidal arteriosclerosis, epiretinal membrane, radiation retinopathy, posterior uveitis, pathologic myopia, and ocular cancer. In some embodiments, the subject to be treated according to the invention is a human.

In some embodiments, the MDM2 inhibitor is delivered intraocularly. For example, in some embodiments, the MDM2 inhibitor is delivered intravitreally.

In another aspect of the present invention is provided a pharmaceutical composition comprising one or more MDM2 inhibitors and one or more ophthalmologically acceptable excipients, the composition being adapted for intraocular delivery. For example, in certain embodiments, the composition is formulated for intravitreal delivery.

In some embodiments, the pharmaceutical composition or the method of treatment includes another compound recognized as effective in the inhibition of cellular proliferation. For example, in certain embodiments, the pharmaceutical composition further comprises an anti-VEGF drug or the method of treatment includes co-administration of an anti-VEGF drug.

### BRIEF DESCRIPTION OF THE DRAWINGS

FIG. 1A is a graph relating to Nutlin-3A in various concentrations, which was added to proliferating HUVECs for 36 hours;

FIG. 1B is a graph relating to Nutlin-3B in various concentrations, which was added to proliferating HUVECs for 36 hours;

FIG. 1C is a graph relating to Nutlin-3A and Nutlin-3B, in various concentrations, which was added to serum free, unchallenged HUVECs;

FIG. 1D (top) is 100× magnified, phase contrast images of representative conditions of serum free, unchallenged HUVECs treated with vehicle (left panel), Nutlin-3B (middle panel), and Nutlin-3A (right panel) at 36 hours and FIG. 1D (bottom) is 200× magnified confocal immunofluorescence images characterizing HUVSMC, indicating that these cells are vimentin (left panel) and smooth muscle actin (middle panel) positive but VE-Cadherin (right panel) negative;

FIG. 1E is a graph generated when Nutlin-3B 7.5 μM, or Nutlin-3A 7.5 μM was added to proliferating HUVSMC at various time points,

FIG. 1F is a graph where HUVSMCs were challenged with FGF-2 and with 5% FBS;

FIG. 1G is a graph where various concentrations (0, 7.5, 15, 30 μM) of Nutlin-3A were added to cultures of serum free, unchallenged HUVSMC for 24 hours;

FIG. 1H is representative images (100× magnification) of serum free HUVSMCs supplemented with FGF-2 after 72 hours of culture;

FIG. 2A illustrates HUVECs that were seeded on plastic bottom culture dishes pre-coated with gelatin and treated with either 5 μM of Nutlin-3A, 5 μM of Nutlin-3B, or vehicle after 8 hours, where images were taken with an epiflourescent microscope and are representative of p53 expression (middle column) in the nucleus (left column) of HUVECs;

FIG. 2B shows Western blot analysis for p53 and p21 performed on lysates obtained from HUVECs treated with Nutlin-3A, Nutlin-3B, or vehicle in various concentrations for 8 hours, where equal amounts of protein lysates were used demonstrated by beta actin;

FIG. 2C is representative 200× magnified confocal images of HUVSMC treated with either 7.5 μM of Nutlin-3A or vehicle after 8 hours (in the color version of this figure, nuclear p53 expression is shown in red and Topro-3, a nuclear stain, is imaged in blue), showing increased p53 expression in the Nutlin-3A treated cells compared to the control, and these cells co-localize to the less intense nuclear marker Topro-3 (hyperintenese cells, indicating p53 expression in the right panel, are not observed on the panel on the left);

FIG. 2D shows Western blot analysis for p53 and p21 performed on lysates obtained from HUVSMCs treated with Nutlin-3A or 3B or vehicle in various concentrations for 24 hours;

FIG. 3A is representative plots from three independent experiments run in duplicate after staining HUVECs with annexin V and propidium iodide, analyzed with flow cytometery, where HUVECs were cultured in growth medium (left panel), vehicle (middle panel) or 7.5 μM of Nutlin-3A (right panel) for 8 hours;

FIG. 3B are representative 200× magnified images of HUVECs grown on culture dishes, wherein HUVECs were conditioned with growth medium (negative control) (upper left), vehicle (lower left), Nutlin-3A 7.5 μM (upper right), or Etoposide 10 μM (positive control) for 24 hours prior to TUNEL staining;

FIG. 3C illustrates the ratio of the number of TUNEL positive cells to the number of nuclei found in 5 random fields from each condition, counted by 2 masked observers, wherein data are mean+/−SD and represent 2 separate experiments;

FIG. 3D shows quantitative RT-PCR on RNA lysates of HUVECs treated with 7.5 μM of Nutlin-3A, 7.5 μM of Nutlin-3B, or vehicle for 4 hours, where relative BAX and BCL-2 expression are presented as a ratio, and values are provided as a percentage of DMSO (control) and expressed as a mean+/−SD (n=9 from 3 independent experiments),*p<0.005;

FIG. 3E shows representative plots from three independent experiments run in duplicate of annexin V and propidium iodide analyzed with flow cytometery of HUVSMCs, wherein HUVSMC were cultured in growth medium (left panel), vehicle (middle panel) or 7.5 μM of Nutlin-3A (right panel) for 36 hours;

FIG. 3F illustrates quantitative RT-PCR on RNA lysates of HUVSMCs treated with 7.5 μM of Nutlin-3A, 7.5 μM of Nutlin-3B or vehicle for 4 hours, with relative BAX and BCL-2 expression are presented as a ratio and values given as a percentage of DMSO (control)+/−SD (n=12 from 4 independent experiments) NS, p>0.05;

FIG. 4A illustrates cell lysates for Western blot analysis used to confirm knock down of p53 in siRNA infected HUVECs (where data are presented as mean±SC, * p<0.05, and scale bars equal 500 mM unless otherwise specified);

FIG. 4B shows p53 siRNA and control siRNA infected HUVECs seeded at 1×10⁶ cells in 6 well plates and incubated with FGF-2 and either Nutlin-3A 7.5 μM or vehicle (DMSO) for 48 hours, where cell proliferation was measured at 48 hours by manual counting using trypan blue exclusion (data are expressed as a mean+/−SD, *p<0.05);

FIG. 5A is images taken with an inverted light microscope, and representative of capillary tube formation at 24 hours (100× magnification), where HUVECs seeded on Matrigel matrix were incubated in the presence of FGF-2 and 7.5 μM of Nutlin-3A, 5 μM of Nutlin-3A, 7.5 μM of Nutlin-3B, or vehicle (DMSO);

FIG. 5B is quantification of Nutlin-3A mediated capillary tube formation inhibition, where results are expressed as a ratio of tubule length measured to total area examined+/−SEM (*p<0.05);

FIG. 6A illustrates postnatal mouse retinal vascular development after birth (upper left panel), and images from left to right show radial growth pattern of post-natal development of retinal vasculature;

FIG. 6B shows that the retinal vasculature is abrogated in the Nutlin-3 treated eyes (n=6) (bottom row) compared to the sham injected mice (n=4) (middle row), and that there is a loss of smaller caliber vessels (inset, right column) in the Nutlin-3 treated mice.

FIGS. 6C and D are graphs representing retinal vasculature measured as a function of pixels compared to the amount of retinal tissue in each mouse eye (*p<0.05, representative of two independent experiments);

FIG. 6E illustrates that Nutlin-3 (lower row) treated mice (n=8) have less retinal vasculature compared to sham injected mice (n=8) (upper row);

FIG. 6F is a 400× magnification of TUNEL positive cells found in the retinal vasculature of a Nutlin-3 treated mouse, with a scale bar of 10 μM;

FIG. 7A is confocal images of GS-IB4 lectin stained retinal vasculature five days after injection;

FIG. 7B is hematxoylin and eosin stained paraffin embedded sections showing normal retinal architecture after sham and Nutlin-3 treated injection; and

FIG. 7C is quantification of retinal vessels, showing no difference between Nutlin-3 (n=8) and sham injected mice (n=8) (NS, p>0.05, summation of two independent experiments).

### DETAILED DESCRIPTION OF THE INVENTION

Many modifications and other embodiments of the inventions set forth herein will come to mind to one skilled in the art to which these inventions pertain having the benefit of the teachings presented herein. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. As used in the specification, and in the appended claims, the singular forms “a”, “an”, “the”, include plural referents unless the context clearly dictates otherwise.

The present invention provides methods for the prevention and/or treatment of unwanted cellular proliferation in the eye. It also provides pharmaceutical compositions comprising one or more MDM2 inhibitors that may be used for the prevention and/or treatment of unwanted cellular proliferation in the eye.

The specific pharmaceutical composition (or compositions) used in the invention, and the methods of treatment provided by the invention, are further described below.

### DEFINITIONS

The term “alkyl” as used herein means saturated straight, branched, or cyclic hydrocarbon groups. In particular embodiments, alkyl refers to groups comprising 1 to 10 carbon atoms (“C1-10 alkyl”). In further embodiments, alkyl refers to groups comprising 1 to 8 carbon atoms (“C1-8 alkyl”), 1 to 6 carbon atoms (“C1-6 alkyl”), 1 to 4 carbon atoms (“C1-4 alkyl”), or 1 to 3 carbon atoms (“C1-3 alkyl”). In other embodiments, alkyl refers to groups comprising 3-10 carbon atoms (“C3-10 alkyl”), 3-8 carbon atoms (“C3-8 alkyl”), or 3-6 carbon atoms (“C3-6 alkyl”). In specific embodiments, alkyl refers to methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. Substituted alkyl includes alkyl substituted with one or more moieties selected from the group consisting of halo (e.g., Cl, F, Br, and I); halogenated alkyl (e.g., CF₃, 2-Br-ethyl, CH₂F, CH₂Cl, CH₂CF₃, or CF₂CF₃); hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “lower alkyl” as used herein means C1-C6 alkyl groups and includes methyl, ethyl, propyl, isopropyl, butyl, t-butyl, 2-butyl, pentyl, hexyl, and the like. Lower alkyl is preferably C1-C4 alkyl, and more preferably C1-C3 alkyl.

The term “alkoxy” as used herein means straight or branched chain alkyl groups linked by an oxygen atom (i.e., —O-alkyl), wherein alkyl is as described above. In particular embodiments, alkoxy refers to oxygen-linked groups comprising 1 to 10 carbon atoms (“C1-10 alkoxy”). In further embodiments, alkoxy refers to oxygen-linked groups comprising 1 to 8 carbon atoms (“C1-8 alkoxy”), 1 to 6 carbon atoms (“C1-6 alkoxy”), 1 to 4 carbon atoms (“C1-4 alkoxy”) or 1 to 3 carbon atoms (“C1-3 alkoxy”).

The term “lower alkoxy” as used herein means lower alkyl groups linked by an oxygen atom (i.e., —O-lower alkyl), wherein lower alkyl is as described above.

The term “alkenyl” as used herein means alkyl moieties wherein at least one saturated CC bond is replaced by a double bond. In particular embodiments, alkenyl refers to groups comprising 2 to 10 carbon atoms (“C2-10 alkenyl”). In further embodiments, alkenyl refers to groups comprising 2 to 8 carbon atoms (“C2-8 alkenyl”), 2 to 6 carbon atoms (“C2-6 alkenyl”), or 2 to 4 carbon atoms (“C2-4 alkenyl”). In specific embodiments, alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl.

The term “amino” as used herein means a moiety represented by the structure NR₂, and includes primary amines, and secondary and tertiary amines substituted by alkyl (i.e., alkylamino). Thus, R₂ may represent, for example, two hydrogen atoms, two alkyl moieties, or one hydrogen atom and one alkyl moiety.

The term “aryl” as used herein means a stable monocyclic, bicyclic, or tricyclic carbon ring of up to 8 members in each ring, wherein at least one ring is aromatic as defined by the Hückel 4n+2 rule.

The term “heteroaryl” as used herein means an aryl group containing from one or more (particularly one to four) non-carbon atom(s) (particularly N or S) or a combination thereof, which heteroaryl group is optionally substituted at one or more carbon or nitrogen atom(s) with alkyl, —CF₃, phenyl, benzyl, or thienyl, or a carbon atom in the heteroaryl group together with an oxygen atom form a carbonyl group, or which heteroaryl group is optionally fused with a phenyl ring. Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5 membered heteroaryls having two heteroatoms in 1, 2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms. “Substituted heteroaryl” means a heteroaryl having one or more non-interfering groups as substituents.

“Substituted” or “optionally substituted” in reference to a substituent group refers to substituent groups optionally substituted with one or more moieties, for example, those selected from the group consisting of optionally substituted C1-10 alkyl (e.g., optionally substituted C1-6 alkyl); optionally substituted C1-10 alkoxy (e.g., optionally substituted C1-6 alkoxy); optionally substituted C2-10 alkenyl; optionally substituted C2-10 alkynyl; optionally substituted C6-C12 aryl; aryloxy; optionally substituted heteroaryl; optionally substituted heterocycle; halo (e.g., Cl, F, Br, and I); hydroxyl; halogenated alkyl (e.g., CF₃, 2-Br-ethyl, CH₂F, CH₂CF₃, and CF₂CF₃); amino (e.g., NH₂, NR₁₂H, and NR₁₂R₁₃); alkylamino; arylamino; acyl; amido; CN; NO₂; N₃; CH₂OH; CONH₂; CONR₁₂R₁₃; CO₂R₁₂; CH₂OR₁₂; NHCOR₁₂; NHCO₂R₁₂; C1-3 alkylthio; sulfate; sulfonic acid; sulfonate esters such as alkyl or aralkyl sulfonyl, including methanesulfonyl; phosphonic acid; phosphate; phosphonate; mono-, di-, or triphosphate esters; trityl or monomethoxytrityl; R₁₂SO; R₁₂SO₂; CF₃S; and CF₃SO₂; trialkylsilyl such as dimethyl-t-butylsilyl or diphenylmethylsilyl; and R₁₂ and R₁₃ are each independently selected from H or optionally substituted C1-10 alkyl.

The term “analogue,” used interchangeably with the term “analog” herein, means a compound in which one or more individual atoms or functional groups have been replaced, either with a different atom or a different functional, generally giving rise to a compound with similar properties.

The term “derivative” as used herein means a compound that is formed from a similar, beginning compound by attaching another molecule or atom to the beginning compound. Further, derivatives, according to the invention, encompass one or more compounds formed from a precursor compound through addition of one or more atoms or molecules or through combining two or more precursor compounds.

The term “prodrug” as used herein means any compound which, when administered to a mammal, is converted in whole or in part to a compound of the invention.

The term “active metabolite” as used herein means a physiologically active compound which results from the metabolism of a compound of the invention, or a prodrug thereof, when such compound or prodrug is administered to a mammal.

The terms “therapeutically effective amount” or “therapeutically effective dose” as used herein are interchangeable and mean a concentration of a compound according to the invention, or a biologically active variant thereof, sufficient to elicit the desired therapeutic effect according to the methods of treatment described herein.

The term “pharmaceutically acceptable carrier” as used herein means a carrier that is conventionally used in the art to facilitate the storage, administration, and/or the healing effect of a biologically active agent.

The term “intermittent administration” as used herein means administration of a therapeutically effective dose of a composition according to the invention, followed by a time period of discontinuance, which is then followed by another administration of a therapeutically effective dose, and so forth.

### Active Agents

The present invention provides methods of treatment of various conditions using certain MDM2-inhibiting compounds and pharmaceutical compositions as well as specific compositions for use according to these methods. The term “MDM2” (Murine Double Minute2) is used herein to mean a protein obtained as a result of expression of the mdm2 gene. Within the meaning of this term, MDM2 encompasses all proteins encoded by mdm2, mutants thereof, alternative slice proteins thereof, and phosphorylated proteins thereof. Additionally, as used herein, the term “MDM2” includes MDM2 analogues, e.g. MDMX, also known as MDM4, and MDM2 homologues and analogues of other animals, e.g. the human homologue HDM2 or the human analogue HDMX.

“MDM2 inhibitor,” as used herein, encompasses any compound that inhibits the activity of MDM2 or its analogues to any extent, particularly inhibition activity that impacts ocular cellular proliferation. In certain preferred embodiments, the MDM2 inhibitor binds to the p53 binding site of MDM2 and may thus affect the ability of MDM2 to interact with p53. For example, compounds that are well-known in the art for blocking the interaction between MDM2 and p53 and may be particularly useful according to the present invention include, but are not limited to, cis-imidazolines (e.g., nutlin compounds), spirooxindoles, diazepines and benzodiazepines (including 1,4-diazepines and 1,4-benzodiazepines), and/or bisarylsulfonamides. Other MDM2 inhibitors of use in accordance with the instant methods can be identified in screening assays for test agents that inhibit the binding of MDM2 to p53.

In certain embodiments, the MDM2 inhibitor comprises a nutlin compound. “Nutlin compound” as used herein, encompasses any cis-imidazoline-based compound. For example, it encompasses Nutlin-1, Nutlin-2, and Nutlin-3, as described in Vassilev, L. T, et al., Science 303 (5659): 844-848 (2004), incorporated herein by reference in its entirety. However, “nutlin compound” is not limited to these compounds; for example, the term “nutlin” further comprises any compound disclosed in U.S. Pat. No. 6,617,346, any compound disclosed in U.S. Pat. No. 6,734,302, and any compound disclosed in U.S. Pat. No. 7,705,007, each patent incorporated herein by reference in its entirety. Additionally, in some embodiments, compounds such as those disclosed in U.S. Patent Application Publication No. 2005/0282803 and U.S. Patent Application Publication No. 2007/0129416, both incorporated herein by reference, are useful according to the present invention.

In preferred embodiments, nutlin compounds useful according to the present invention comprise a cis imidazoline substituted at the 4 and 5 positions with optionally substituted phenyl rings. In preferred embodiments, the compounds comprise a cis imidazoline substituted at the 2 position with an optionally substituted phenyl, pyridinyl, pyrimidinyl, pyrazinyl, pyridazinyl, or triazinyl ring. In other preferred embodiments, the compounds comprise a cis imidazoline substituted at the 2 position with an optionally substituted thiophene.

In some embodiments, nutlin compounds according to the present invention include, but are not limited to, compounds according to the following structure:

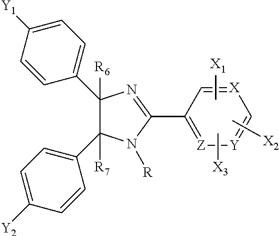

wherein:

R is H or —C═OR₁;

R₁ is lower alkyl, cycloalkyl, —C═CHCOOH, —CH₂CH₂Ph, 2-furanyl, phenyl, phenyl substituted with Cl, OCH₃, or cyano; amino, —NHCH₂CH₂R₂, —N(CH₂CH₂OH)CH₂CH₂OH, —N(CH₃)CH₂CH₂NCH₃, —N(CH₃)CH₂CH₂N(CH₃)CH₃, saturated 4-, 5- and 6-membered rings, saturated and unsaturated 5- and 6-membered rings containing at least one hetero atom wherein the hetero atom is selected from S, N and O and being optionally substituted with a group selected from lower alkyl, —C═O—R₅, —OH, lower alkyl substituted with hydroxy, lower alkyl substituted with —NH₂, —N(CH₃)CH₃, N-lower alkyl, —N—X₈X₉, —SO₂CH₃, ═O, —C═OCH₃, —CH₂C═OCH₃, and 5- and 6-membered saturated or unsaturated rings containing at least one hetero atom selected from S, N and O;

R₂ is selected from —N(CH₃)CH₃, —NHCH₂CH₂NH₂, —NH₂, morpholinyl, and piperazinyl;

X, Y, and Z are independently selected from C and N;

X₁, X₂ and X₃ are independently selected from —H, —OH, lower alkyl, lower alkoxy, lower alkoxy substituted with F or trifluoromethyl, —Cl, —Br, —F, —CH₂OCH₃, —CH₂OCH₂CH₃, CF₃, OCH₂CH₂R₃, —CH₂-morpholino, —CH₂CF₃, —OCH₂CF₃, —OCH(CH₃)CH₂OH, —OR₄, —CH₂R₄, —COOQ, —SCH₃, —NO₂, —N(CH₃)₂, —OCH₂-phenyl, —OCH₂C═OOQ, —C(X₄X₅)—X₆, or a saturated 5- or 6-membered ring containing at least one hetero atom wherein the hetero atom is selected from S, N, and O; or one of X₁, X₂ or X₃ is H and the other two taken together with the two carbon atoms and the bonds between them from the benzene ring to which they are substituted form a 5- or 6-membered saturated or unsaturated ring or a 5- or 6-membered saturated or unsaturated ring containing at least one hetero atom wherein the hetero atom is selected from S, N, and O;

R₃ is selected from —F, —OCH₃, —N(CH₃)CH₃, and unsaturated 5-membered rings containing at least one hetero atom wherein the hetero atom is selected from S, N, and O;

R₄ is a 3- to 6-membered saturated ring;

R₅ is selected from H, lower alkyl, —NH₂, —N-lower alkyl, lower alkyl substituted with hydroxy, and lower alkyl substituted with NH₂;

R₆ and R₇ are independently selected from H, CH₃, CH₂CH₃, CH₂OH, and CH₂OCH₃;

Q is H, —NH₂, or lower alkyl;

X₄ and X₅ are lower alkyl and can be connected together to form a cycloalkyl;

X₆ is selected from the group consisting of lower alkyl, cyano, —CH₂OH, —CH₂O-lower alkyl, —CH₂O-lower alkyl substituted by lower alkoxy, —C(O)X₇, and CH₂NX₈X₉;

X₇ is selected from the group consisting of hydroxy, lower alkoxy, morpholino, and —NX₈X₉;

X₈ and X₉ are independently selected from the group consisting of H, lower alkyl, lower alkyl substituted by lower alkoxy or cyano, and lower alkoxy; and

Y₁ and Y₂ are each independently selected from —Cl, —Br, —NO₂, trifluoromethyl, —C≡N, and —C≡CH;

and pharmaceutically acceptable salts and esters thereof.

In certain embodiments, one of X₁, X₂ or X₃ is H and the other two are independently selected from hydroxy, lower alkyl, lower alkoxy, Cl, Br, F, CF₃, —CH₂OCH₃, —CH₂OCH₂CH₃—OCH₂CH₂R₃, —CH₂-morpholino, —CH₂CF₃, —OCH₂CF₃, —OCH(CH₃)CH₂OH, —OR₄, —CH₂R₄, and COOQ.

In one preferred embodiment, the present invention relates to the use of Nutlin-3 ((±)-4-[4,5-bis(4-chlorophenyl)-2-(2-isopropoxy-4-methoxy-phenyl)-4,5-dihydro-imidazole-1-carbonyl]-piperazin-2-one).

In other embodiments, the present invention relates to the use of the following specific nutlin compounds: 1-[4,5-Bis-(4-chloro-phenyl)-2-(2,4-dimethoxy-phenyl)-4,5-dihydro-imidazol-1-yl]-2-methyl-propan-1-one; 1-[4,5-Bis-(4-chloro-phenyl)-2-(2,4-dimethoxy-phenyl)-4,5-dihydro-imidazol-1-yl]ethanone; 1-[4,5-Bis-(4-chloro-phenyl)-2-(2,4-dimethoxy-phenyl)-4,5-dihydro-imidazol-1-yl]-2,2-dimethyl-propan-1-one; [4,5-Bis-(4-chloro-phenyl)-2-(2,4-dimethoxy-phenyl)-4,5-dihydro-imidazol-1-yl]-cyclopentyl-methanone; [4,5-Bis-(4-chloro-phenyl)-2-(2,4-dimethoxy-phenyl)-4,5-dihydro-imidazol-1-yl]-cyclohexyl-methanone; [4,5-Bis-(4-chloro-phenyl)-2-(2,4-dimethoxy-phenyl)-4,5-dihydro-imidazol-1-yl]-thiophen-2-yl-methanone; [4,5-Bis-(4-chloro-phenyl)-2-(2,4-dimethoxy-phenyl)-4,5-dihydro-imidazol-1-yl]-isoxazol-5-yl-methanone; [4,5-Bis-(4-chloro-phenyl)-2-(2,4-dimethoxy-phenyl)-4,5-dihydro-imidazol-1-yl]-furan-2-yl-methanone; 1-[4,5-Bis-(4-chloro-phenyl)-2-(2,3-dimethoxy-phenyl)-4,5-dihydro-imidazol-1-yl]-2-methyl-propan-1-one; and/or [4,5-Bis-(4-chloro-phenyl)-2-(2,4-dimethoxy-phenyl)-4,5-dihydro-imidazol-1-yl]-(4-methyl-piperazin-1-yl)-methanone.

In other embodiments, the present invention relates to the use of the following specific nutlin compounds: 1-{4-[4,5-Bis-(4-bromo-phenyl)-2-(2-isopropoxy-4-methoxy-phenyl)-4,5-dihydro-imidazole-1-carbonyl]-piperazin-1-yl}-ethanone; [4,5-Bis-(4-bromo-phenyl)-2-(2-isopropoxy-4-methoxy-phenyl)-4,5-dihydro-imidazol-1-yl]-(4-methanesulfonyl-piperazin-1-yl)-methanone; [4,5-Bis-(4-bromo-phenyl)-2-(2-isopropoxy-4-methoxy-phenyl)-4,5-dihydro-imidazol-1-yl]-(4-methyl-piperazin-1-yl)-methanone; [4,5-Bis-(4-bromo-phenyl)-2-(2-isopropoxy-4-methoxy-phenyl)-4,5-dihydro-imidazol-1-yl]-morpholin-4-yl-methanone; [1,4′]Bipiperidinyl-1′-yl-[4,5-bis-(4-bromo-phenyl)-2-(2-isopropoxy-4-methoxy-phenyl)-4,5-dihydro-imidazol-1-yl]-methanone; [4,5-Bis-(4-bromo-phenyl)-2-(2-isopropoxy-4-methoxy-phenyl)-4,5-dihydro-imidazol-1-yl]-(4-ethyl-piperazin-1-yl)-methanone; 4-[4,5-Bis-(4-bromo-phenyl)-2-(2-isopropoxy-4-methoxy-phenyl)-4,5-dihydro-imidazole-1-carbonyl]-piperazin-2-one; [4,5-Bis-(4-cyano-phenyl)-2-(2-isopropoxy-4-methoxy-phenyl)-4,5-dihydro-imidazol-1-yl]-piperazin-1-yl-methanone; 1-(4-{4,5-Bis-(4-chloro-phenyl)-2-[4-methoxy-2-(2-methoxy-ethoxy)-phenyl]-4,5-dihydro-imidazole-1-carbonyl}-piperazin-1-yl)-ethanone; and/or 1-(4-{4,5-Bis-(4-chloro-phenyl)-2-[2-(2-fluoro-ethoxy)-4-methoxy-phenyl]-4,5-dihydro-imidazole-1-carbonyl}-piperazin-1-yl)-ethanone;

In other embodiments, the present invention relates to the use of the following specific nutlin compounds: [4,5-Bis-(4-chloro-phenyl)-2-(2-ethoxy-4-fluoro-phenyl)-4,5-dihydro-imidazol-1-yl]-(4-pyrrolidin-1-yl-piperazin-1-yl)-methanone; [4,5-Bis-(4-chloro-phenyl)-2-(2-ethoxy-4-fluoro-phenyl)-4,5-dihydro-imidazol-1-yl]-(4-dimethylamino-piperazin-1-yl)-methanone; [4,5-Bis-(4-chloro-phenyl)-2-(2-ethoxy-4-fluoro-phenyl)-4,5-dihydro-imidazol-1-yl]-piperazin-1-yl)-methanone; [4,5-Bis-(4-chloro-phenyl)-2-(4-fluoro-2-isopropoxy-phenyl)-4,5-dihydro-imidazol-1-yl]-(4-pyrrolidin-1-yl-piperazin-1-yl)-methanone; [4,5-Bis-(4-chloro-phenyl)-2-(4-fluoro-2-isopropoxy-phenyl)-4,5-dihydro-imidazol-1-yl]-(4-dimethylamino-piperazin-1-yl)-methanone; [4,5-Bis-(4-chloro-phenyl)-2-(4-fluoro-2-isopropoxy-phenyl)-4,5-dihydro-imidazol-1-yl]-(4-methyl-piperazin-1-yl)-methanone; [4,5-Bis-(4-chloro-phenyl)-2-(4-fluoro-2-isopropoxy-phenyl)-4,5-dihydro-imidazol-1-yl]-piperazin-1-yl)-methanone; [4,5-Bis-(4-chloro-phenyl)-2-(4-fluoro-2-isopropoxy-phenyl)-4,5-dihydro-imidazol-1-yl]-(4-methanesulfonyl-piperazin-1-yl)-methanone; 4-[4,5-Bis-(4-chloro-phenyl)-2-(4-fluoro-2-isopropoxy-phenyl)-4,5-dihydro-imidazol-1-carbonyl]-piperazin-2-one; and/or [4,5-Bis-(4-chloro-phenyl)-2-chroman-8-yl-4,5-dihydro-imidazol-1-yl]-piperazin-1-yl-methanone.

In further embodiments, the present invention relates to the use of the following specific nutlin compounds: [4,5-Bis-(4-chloro-phenyl)-2-(2-isopropoxy-4-methoxy-phenyl)-4,5-dihydro-imidazol-1-yl]-[4-(2-hydroxy-propyl)-piperazin-1-yl]-methanone; [4,5-Bis-(4-chloro-phenyl)-2-(2-isopropoxy-4-methoxy-phenyl)-4,5-dihydro-imidazol-1-yl]-threo[4-(2-hydroxy-1-methyl-propyl)-piperazin-1-yl]-methanone; [4,5-Bis-(4-chloro-phenyl)-2-(2-isopropoxy-4-methoxy-phenyl)-4,5-dihydro-imidazol-1-yl]-erythro[4-(2-hydroxy-1-methyl-propyl)-piperazin-1-yl]-methanone; 1-{4-[4,5-Bis-(4-chloro-phenyl)-2-(2-isopropoxy-4-methoxy-phenyl)-4,5-dihydro-imidazole-1-carbonyl]-piperazin-1-yl}-propan-2-one; [4,5-Bis-(4-chloro-phenyl)-2-(2-isopropoxy-4-methoxy-phenyl)-4,5-dihydro-imidazol-1-yl]-[1,4]diazepan-1-yl-methanone; 4-[4,5-Bis-(4-chloro-phenyl)-2-(2-isopropoxy-4-methoxy-phenyl)-4,5-dihydro-imidazole-1-carbonyl]-1-methyl-piperazin-2-one; 1-{4-[4,5-Bis-(4-chloro-phenyl)-2-(2-isopropoxy-4-methoxy-phenyl)-4,5-dihydro-imidazole-1-carbonyl]-piperazin-1-yl}-2-methyl-propan-1-one; 4-[4,5-Bis-(4-chloro-phenyl)-2-(2-isopropoxy-4-methoxy-phenyl)-4,5-dihydro-imidazole-1-carbonyl]-piperazin-1-carbaldehyde; 4-{4,5-Bis-(4-chloro-phenyl)-2-[4-methoxy-2-(2,2,2-trifluoro-ethoxy)-phenyl]-4,5-dihydro-imidazole-1-carbonyl}-piperazin-2-one; 4-{4,5-Bis-(4-bromo-phenyl)-2-[4-methoxy-2-(2,2,2-trifluoro-ethoxy)-phenyl]-4,5-dihydro-imidazole-1-carbonyl}-piperazin-2-one; [4,5-Bis-(4-ethynyl-phenyl)-2-(2-isopropoxy-4-methoxy-phenyl)-4,5-dihydro-imidazol-1-yl]-(4-pyrrolidin-1-yl-piperidin-1-yl)-methasone; 1-{4-2-(5-Chloro-2-isopropoxy-phenyl)-4,5-bis-(4-chloro-phenyl)-4,5-dihydro-imidazole-1-carbonyl]-piperazin-1-yl}-ethanone; and/or [5-(4-Chloro-phenyl)-4-(4-ethynyl-phenyl)-2-(2-isopropoxy-4-methoxy-phenyl)-4,5-dihydro-imidazole-1-yl]-(4-pyrrolidin-1-yl-piperidin-1-yl)-methanone;

In still further embodiments, the present invention relates to the use of the following specific nutlin compounds: 1-[4,5-Bis-(4-chloro-phenyl)-2-(2-methoxy-phenyl)-4,5-dihydro-imidazol-1-yl]-2-methyl-propan-1-one; 1-[4,5-Bis-(4-chloro-phenyl)-2-p-tolyl-4,5-dihydro-imidazol-1-yl]-ethanone; {4-[4,5-Bis-(4-chloro-phenyl)-1-isobutyryl-4,5-dihydro-1H-imidazol-2-yl]-phenoxy}-acetic acid ethyl ester; {4-[4,5-Bis-(4-chloro-phenyl)-1-isobutyryl-4,5-dihydro-1H-imidazol-2-yl]-phenoxy}-acetic acid; 2-Methyl-1-[2,4,5-tris-(4-chloro-phenyl)-4,5-dihydro-imidazol-1-yl]-propan-1-one; 1-[4,5-Bis-(4-chloro-phenyl)-2-(4-methoxy-phenyl)-4,5-dihydro-imidazol-1-yl]-ethanone; [2-(2-Chloro-phenyl)-4,5-bis-(4-chloro-phenyl)-4,5-dihydro-imidazol-1-yl]-piperazin-1-yl-methanone; [2-(3-Bromo-phenyl)-4,5-bis-(4-chloro-phenyl)-4,5-dihydro-imidazol-1-yl]-piperazin-1-yl-methanone; [2-Biphenyl-3-yl-4,5-bis-(4-chloro-phenyl)-4,5-dihydro-imidazol-1-yl]-(4-methyl-piperazin-1-yl-methanone; and/or [4,5-Bis-(4-chloro-phenyl)-2-(3-pyrrolidin-1-yl-phenyl)-4,5-dihydro-imidazol-1-yl]-piperazin-1-yl-methanone.

In other embodiments, the present invention relates to the use of the following specific nutlin compounds: [4,5-Bis-(4-chloro-phenyl)-2-(2,4-dimethoxy-phenyl)-4,5-dihydro-imidazol-1-yl]-piperazin-1-yl-methanone; [4,5-Bis-(4-chloro-phenyl)-2-(2-fluoro-6-methoxy-phenyl)-4,5-dihydro-imidazol-1-yl]-[4-(2-hydroxy-ethyl)-piperazin-1-yl]-methanone; 1-{4-[4,5-Bis-(4-bromo-phenyl)-2-(2,4-dimethoxy-phenyl)-4,5-dihydro-imidazole-1-carbonyl]-piperazin-1-yl}-ethanone; [4,5-Bis-(4-bromo-phenyl)-2-(2,4-dimethoxy-phenyl)-4,5-dihydro-imidazol-1-yl]-[4-(2-hydroxy-ethyl)-piperazin-1-yl]-methanone; [4,5-Bis-(4-chloro-phenyl)-2-(2-isopropoxy-4-methoxy-phenyl)-4,5-dihydro-imidazol-1-yl]-[4-(2-hydroxy-ethyl)-piperazin-1-yl]-methanone hydrochloride; [4,5-Bis-(4-chloro-phenyl)-2-(2-ethoxy-4-methoxy-phenyl)-4,5-dihydro-imidazol-1-yl]-(4-methanesulfonyl-piperazin-1-yl)-methanone; [4,5-Bis-(4-chloro-phenyl)-2-(2-ethoxy-4-methoxy-phenyl)-4,5-dihydro-imidazol-1-yl]-[4-(2-hydroxy-ethyl)-piperazin-1-yl]-methanone; [4,5-Bis-(4-chloro-phenyl)-2-(2-ethoxy-4-methoxy-phenyl)-4,5-dihydro-imidazol-1-yl]-morpholin-4-yl-methanone; [4,5-Bis-(4-chloro-phenyl)-2-(2-ethoxy-4-methoxy-phenyl)-4,5-dihydro-imidazol-1-yl]-(4-methyl-piperazin-1-yl)-methanone; and/or 1-{4-[4,5-Bis-(4-chloro-phenyl)-2-(2-ethoxy-4-methoxy-phenyl)-4,5-dihydro-imidazole-1-carbonyl]-piperazin-1-yl}-ethanone.

In other embodiments, the present invention relates to the use of the following specific nutlin compounds: 4-[4,5-Bis-(4-chloro-phenyl)-2-(2-ethoxy-4-methoxy-phenyl)-4,5-dihydro-imidazole-1-carbonyl]-piperazin-2-one; [4,5-Bis-(4-chloro-phenyl)-2-(2-isopropoxy-4-methoxy-phenyl)-4,5-dihydro-imidazol-1-yl]-piperazin-1-yl-methanone; 1-{4-[4,5-Bis-(4-chloro-phenyl)-2-(2-isopropoxy-4-methoxy-phenyl)-4,5-dihydro-imidazole-1-carbonyl]-piperazin-1-yl}-ethanone; [4,5-Bis-(4-chloro-phenyl)-2-(2-isopropoxy-4-methoxy-phenyl)-4,5-dihydro-imidazole-1-yl]-(4-methanesulfonyl-piperazin-1-yl)-methanone; [4,5-Bis-(4-chloro-phenyl)-2-(2-isopropoxy-4-methoxy-phenyl)-4,5-dihydro-imidazol-1-yl]-(2,5-dimethyl-piperazin-1-yl)-methanone; 4,5-Bis-(4-chloro-phenyl)-2-(2-isopropoxy-4-methoxy-phenyl)-4,5-dihydro-imidazole-1-carboxylic acid bis-(2-hydroxy-ethyl)-amide; [4,5-Bis-(4-chloro-phenyl)-2-(2-isopropoxy-4-methoxy-phenyl)-4,5-dihydro-imidazol-1-yl]-(4-ethyl-piperazin-1-yl)-methanone; [1,4′]Bipiperidinyl-1′-yl-[4,5-bis-(4-chloro-phenyl)-2-(2-isopropoxy-4-methoxy-phenyl)-4,5-dihydro-imidazol-1-yl]-methanone; [4,5-Bis-(4-chloro-phenyl)-2-(2-isopropoxy-4-methoxy-phenyl)-4,5-dihydro-imidazol-1-yl]-(4-pyrrolidin-1-yl-piperidin-1-yl)-methanone; and/or [4,5-Bis-(4-chloro-phenyl)-2-(2-isopropoxy-4-methoxy-phenyl)-4,5-dihydro-imidazol-1-yl]-(4-dimethylamino-piperidin-1-yl)-methanone.

In further embodiments, the present invention relates to the use of the following specific nutlin compounds: [4,5-Bis-(4-chloro-phenyl)-2-(2-isopropoxy-4-methoxy-phenyl)-4,5-dihydro-imidazol-1-yl]-morpholin-4-yl-methanone; [4,5-Bis-(4-chloro-phenyl)-2-(2-isopropoxy-4-methoxy-phenyl)-4,5-dihydro-imidazol-1-yl]-(4-isopropyl-piperazin-1-yl)-methanone; 4-[4,5-Bis-(4-chloro-phenyl)-2-(2-isopropoxy-4-methoxy-phenyl)-4,5-dihydro-imidazole-1-carbonyl]-piperazin-2-one; [4,5-Bis-(4-chloro-phenyl)-2-(2-isopropoxy-4-methoxy-phenyl)-4,5-dihydro-imidazol-1-yl]-(4-hydroxymethyl-piperidin-1-yl)-methanone; [4,5-Bis-(4-chloro-phenyl)-2-(2-isopropoxy-4-methoxy-phenyl)-4,5-dihydro-imidazol-1-yl]-[4-(hydroxy-ethyl)-piperidin-1-yl]-methanone; [4,5-Bis-(4-chloro-phenyl)-2-(2-isopropoxy-4-methoxy-phenyl)-4,5-dihydro-imidazol-1-yl]-(3-methyl-piperazin-1-yl)-methanone; 1-{4-[4,5-Bis-(4-chloro-phenyl)-2-(2-isopropoxy-4-methoxy-phenyl)-4,5-dihydro-imidazole-1-carbonyl]-2-methyl-piperazin-1-yl}-ethanone; [4,5-Bis-(4-chloro-phenyl)-2-(2-isopropoxy-4-methoxy-phenyl)-4,5-dihydro-imidazol-1-yl]-(4-methanesulfonyl-3-methyl-piperazin-1-yl)-methanone; [4,5-Bis-(4-chloro-phenyl)-2-(2-isopropoxy-4-methoxy-phenyl)-4,5-dihydro-imidazol-1-yl]-(4-hydroxy-piperidin-1-yl)-methanone; and/or (4-Aminomethyl-piperidin-1-yl)-[4,5-bis-(4-chloro-phenyl)-2-(2-isopropoxy-4-methoxy-phenyl)-4,5-dihydro-imidazol-1-yl]-methanone.

In further embodiments, the present invention relates to the use of the following specific nutlin compounds: [4,5-Bis-(4-bromo-phenyl)-2-(2-ethoxy-4-methoxy-phenyl)-4,5-dihydro-imidazol-1-yl]-[4-(2-hydroxy-ethyl)-piperazin-1-yl]-methanone; [4,5-Bis-(4-bromo-phenyl)-2-(2-ethoxy-4-methoxy-phenyl)-4,5-dihydro-imidazol-1-yl]-piperazin-1-yl-methanone; [4,5-Bis-(4-bromo-phenyl)-2-(2-ethoxy-4-methoxy-phenyl)-4,5-dihydro-imidazol-1-yl]-(4-methanesulfonyl-piperazin-1-yl)-methanone; 1-{4-[4,5-Bis-(4-bromo-phenyl)-2-(2-ethoxy-4-methoxy-phenyl)-4,5-dihydro-imidazole-1-carbonyl]-piperazin-1-yl}-ethanone; [4,5-Bis-(4-bromo-phenyl)-2-(2-ethoxy-4-methoxy-phenyl)-4,5-dihydro-imidazol-1-yl]-(4-methyl-piperazin-1-yl)-methanone; 4-[4,5-Bis-(4-bromo-phenyl)-2-(2-ethoxy-4-methoxy-phenyl)-4,5-dihydro-imidazole-1-carbonyl]-piperazine-1-carbaldehyde; [4,5-Bis-(4-bromo-phenyl)-2-(2-ethoxy-4-methoxy-phenyl)-4,5-dihydro-imidazol-1-yl]-(4-pyrrolidin-1-yl-piperidin-1-yl)-methanone; [4,5-Bis-(4-bromo-phenyl)-2-(2-ethoxy-4-methoxy-phenyl)-4,5-dihydro-imidazol-1-yl]-(4-dimethylamino-piperidin-1-yl)-methanone; [4,5-Bis-(4-bromo-phenyl)-2-(2-ethoxy-4-methoxy-phenyl)-4,5-dihydro-imidazol-1-yl]-(4-isopropyl-piperazin-1-yl)-methanone; 4-[4,5-Bis-(4-bromo-phenyl)-2-(2-ethoxy-4-methoxy-phenyl)-4,5-dihydro-imidazole-1-carbonyl]-piperazin-2-one; and/or [4,5-Bis-(4-bromo-phenyl)-2-(2-isopropoxy-4-methoxy-phenyl)-4,5-dihydro-imidazol-1-yl]-piperazin-1-yl-methanone.

In still further embodiments, the present invention relates to the use of the following specific nutlin compounds: 4-{4,5-Bis-(4-chloro-phenyl)-2-[2-(2-fluoro-ethoxy)-4-methoxy-phenyl]-4,5-dihydro-imidazole-1-carbonyl}-piperazin-2-one; [4,5-Bis-(4-chloro-phenyl)-2-(2-ethoxy-4-methoxy-phenyl)-4,5-dihydro-imidazol-1-yl]-piperazin-1-yl-methanone hydrochloride; 4,5-Bis-(4-chloro-phenyl)-2-(2-ethoxy-4-methoxy-phenyl)-4,5-dihydro-imidazole-1-carboxylic acid methyl-(2-methylamino-ethyl)-amide, trifluoroacetic acid salt; 4,5-Bis-(4-chloro-phenyl)-2-(2-ethoxy-4-methoxy-phenyl)-4,5-dihydro-imidazole-1-carboxylic acid (2-dimethylamino-ethyl)-methyl-amide, trifluoroacetic acid salt; 4,5-Bis-(4-chloro-phenyl)-2-(2-ethoxy-4-methoxy-phenyl)-4,5-dihydro-imidazole-1-carboxylic acid (2-dimethylamino-ethyl)-amide, trifluoroacetic acid salt; 4,5-Bis-(4-chloro-phenyl)-2-(2-ethoxy-4-methoxy-phenyl)-4,5-dihydro-imidazole-1-carboxylic acid (2-amino-ethyl)-amide, trifluoroacetic acid salt; [4,5-Bis-(4-chloro-phenyl)-2-(2-ethoxy-4-methoxy-phenyl)-4,5-dihydro-imidazol-1-yl]-(4-pyrrolidin-1-yl-piperidin-1-yl)-methanone hydrochloride; [4,5-Bis-(4-chloro-phenyl)-2-(4-methoxy-2-propoxy-phenyl)-4,5-dihydro-imidazol-1-yl]-piperazin-1-yl-methanone, trifluoroacetic acid salt; [4,5-Bis-(4-chloro-phenyl)-2-(2-isopropoxy-4-methoxy-phenyl)-4,5-dihydro-imidazol-1-yl]-(4-methyl-piperazin-1-yl)-methanone hydrochloride; and/or 4,5-Bis-(4-chloro-phenyl)-2-(2-isopropoxy-4-methoxy-phenyl)-4,5-dihydro-imidazole-1-carboxylic acid (2-morpholin-4-yl-ethyl)-amide hydrochloride.

In still further embodiments, the present invention relates to the use of the following specific nutlin compounds: 4,5-Bis-(4-chloro-phenyl)-2-(2-isopropoxy-4-methoxy-phenyl)-4,5-dihydro-imidazole-1-carboxylic acid (2-piperazin-1-yl-ethyl)-amide hydrochloride; [4,5-Bis-(4-chloro-phenyl)-2-(2-isobutoxy-4-methoxy-phenyl)-4,5-dihydro-imidazol-1-yl]-piperazin-1-yl-methanone hydrochloride; [4,5-Bis-(4-chloro-phenyl)-2-(2-isopropoxy-4-methoxy-phenyl)-4,5-dihydro-imidazol-1-yl]-(3-methyl-piperazin-1-yl)-methanone hydrochloride; {4,5-Bis-(4-chloro-phenyl)-2-[4-methoxy-2-(2-methoxy-ethoxy)-phenyl]-4,5-dihydro-imidazol-1-yl}-piperazin-1-yl-methanone, trifluoroacetic acid salt; {4,5-Bis-(4-chloro-phenyl)-2-[2-(2-fluoro-ethoxy)-4-methoxy-phenyl]-4,5-dihydro-imidazol-1-yl}-1-piperazin-1-yl-methanone; {4,5-Bis-(4-chloro-phenyl)-2-[2-(2-fluoro-ethoxy)-4-methoxy-phenyl]-4,5-dihydro-imidazol-1-yl-}-(4-pyrrolidin-1-yl-piperidin-1-yl)-methanone hydrochloride; 2-Amino-1-{4-[4,5-bis-(4-chloro-phenyl)-2-(2-isopropoxy-4-methoxy-phenyl)-4,5-dihydro-imidazole-1-carbonyl]-piperazin-1-yl}-ethanone; 1-{4-[4,5-Bis-(4-chloro-phenyl)-2-(2-isopropoxy-4-methoxy-phenyl)-4,5-dihydro-imidazole-1-carbonyl]-piperazin-1-yl}-2-hydroxy-ethanone; 1-{4-[4,5-Bis-(4-chloro-phenyl)-2-(2-isopropoxy-4-methoxy-phenyl)-4,5-dihydro-imidazole-1-carbonyl]-piperazin-1-yl}-2,3-dihydroxy-propan-1-one; and/or [4,5-Bis-(4-chloro-phenyl)-2-(2-isopropoxy-4-methoxy-phenyl)-4,5-dihydro-imidazol-1-yl]-[4-(2,3-dihydroxy-propyl)-piperazin-1-yl]-methanone.

In other embodiments, the present invention relates to the use of the following specific nutlin compounds: 4-[4,5-Bis-(4-chloro-phenyl)-2-(2-isopropoxy-4-methoxy-phenyl)-4,5-dihydro-imidazole-1-carbonyl]-piperazine-1-carboxylic acid dimethylamide; 4-[4,5-Bis-(4-chloro-phenyl)-2-(2-isopropoxy-4-methoxy-phenyl)-4,5-dihydro-imidazole-1-carbonyl]-piperazine-1-carboxylic acid amide; [4,5-Bis-(4-chloro-phenyl)-2-(2,4-diethoxy-phenyl)-4,5-dihydro-imidazol-1-yl]-piperazin-1-yl-methanone; [4,5-Bis-(4-chloro-phenyl)-2-(2,4-diethoxy-phenyl)-4,5-dihydro-imidazol-1-yl]morpholin-4-yl-methanone; [4,5-Bis-(4-chloro-phenyl)-2-(2,4-diethoxy-phenyl)-4,5-dihydro-imidazol-1-yl]-[4-(2-hydroxy-ethyl)-piperazin-1-yl]-methanone; [4,5-Bis-(4-chloro-phenyl)-2-(2,4-diethoxy-phenyl)-4,5-dihydro-imidazol-1-yl]-(4-methyl-piperazin-1-yl)-methanone; 1-{4-[4,5-Bis-(4-chloro-phenyl)-2-(2,4-diethoxy-phenyl)-4,5-dihydro-imidazole-1-carbonyl]-piperazin-1-yl}-ethanone; [4,5-Bis-(4-chloro-phenyl)-2-(2,4-diethoxy-phenyl)-4,5-dihydro-imidazol-1-yl]-(4-methanesulfonyl-piperazin-1-yl)-methanone; [4,5-Bis-(4-chloro-phenyl)-2-(2,4-diethoxy-phenyl)-4,5-dihydro-imidazol-1-yl]-(4-pyrrolidin-1-piperidin-1-yl)-methanone; and/or [4,5-Bis-(4-bromo-phenyl)-2-(2,4-diethoxy-phenyl)-4,5-dihydro-imidazol-1-yl]-(4-methyl-piperazin-1-yl)-methanone.

In other embodiments, the present invention relates to the use of the following specific nutlin compounds: [4,5-Bis-(4-bromo-phenyl)-2-(2,4-diethoxy-phenyl)-4,5-dihydro-imidazol-1-yl]-(4-ethyl-piperazin-1-yl)-methanone; [4,5-Bis-(4-bromo-phenyl)-2-(2,4-diethoxy-phenyl)-4,5-dihydro-imidazol-1-yl]-morpholin-4-yl-methanone; 4-[4,5-Bis-(4-chloro-phenyl)-2-(2,4-diethoxy-phenyl)-4,5-dihydro-imidazole-1-carbonyl]-piperazine-1-carboxylic acid amide; 4-[4,5-Bis-(4-chloro-phenyl)-2-(2,4-diethoxy-phenyl)-4,5-dihydro-imidazole-1-carbonyl]-piperazine-1-carboxylic acid dimethylamide; [4,5-Bis-(4-chloro-phenyl)-2-(4-dimethylamino-2-ethoxy-phenyl)-4,5-dihydro-imidazol-1-yl]-piperazin-1-yl-methanone; [4,5-Bis-(4-chloro-phenyl)-2-(2-ethoxy-4-ethyl-phenyl)-4,5-dihydro-imidazol-1-yl]-piperazin-1-methanone; [4,5-Bis-(4-chloro-phenyl)-2-(2-ethoxy-4-methyl-phenyl)-4,5-dihydro-imidazol-1-yl]-piperazin-1-yl-methanone; [4,5-Bis-(4-chloro-phenyl)-2-(4-ethyl-2-isopropoxy-phenyl)-4,5-dihydro-imidazol-1-yl]-piperazin-1-yl-methanone; [4,5-Bis-(4-chloro-phenyl)-2-(2-isopropoxy-4-methyl-phenyl)-4,5-dihydro-imidazol-1-yl]-piperazin-1-yl-methanone; 1-{4-[4,5-Bis-(4-chloro-phenyl)-2-(4-dimethylamino-2-ethoxy-phenyl)-4,5-dihydro-imidazole-1-carbonyl]-piperazin-1-yl)-ethanone; and/or [4,5-Bis-(4-chloro-phenyl)-2-(2-ethoxy-5-methyl-phenyl)-4,5-dihydro-imidazol-1-yl]-piperazin-1-yl-methanone, trifluoroacetic acid salt.

In further embodiments, the present invention relates to the use of the following specific nutlin compounds: 4-[4,5-Bis-(4-bromo-phenyl)-2-(2-ethoxy-4-fluoro-phenyl)-4,5-dihydro-imidazole-1-carbonyl]-piperazin-2-one; [4,5-Bis-(4-bromo-phenyl)-2-(2-ethoxy-4-fluoro-phenyl)-4,5-dihydro-imidazol-1-yl]-morpholin-4-yl-methanone; [4,5-Bis-(4-bromo-phenyl)-2-(2-ethoxy-4-fluoro-phenyl)-4,5-dihydro-imidazol-1-yl]-(4-pyrrolidin-1-yl-piperidin-1-yl)-methanone; [4,5-Bis-(4-bromo-phenyl)-2-(2-ethoxy-4-fluoro-phenyl)-4,5-dihydro-imidazol-1-yl]-(4-dimethylamino-piperidin-1-yl)-methanone; [4,5-Bis-(4-bromo-phenyl)-2-(2-ethoxy-4-fluoro-phenyl)-4,5-dihydro-imidazol-1-yl]-piperazin-1-methanone; [4,5-Bis-(4-chloro-phenyl)-2-(2-cyclopentyloxy-4-methoxy-phenyl)-4,5-dihydro-imidazol-1-yl]-piperazin-1-yl-methanone; {4,5-Bis-(4-chloro-phenyl)-2-[2-(2-dimethylamino-ethoxy)-4-methoxy-phenyl]-4,5-dihydro-imidazol-1-yl}-piperazin-1-yl-methanone; {4,5-Bis-(4-chloro-phenyl)-2-[2-(2-imidazol-1-yl-ethoxy)-4-methoxy-phenyl]-4,5-dihydro-imidazol-1-yl}-piperazin-1-yl-methanone; [2-(4-Chloro-2-ethoxy-phenyl)-4,5-bis-(4-chloro-phenyl)-4,5-dihydro-imidazol-1-yl]-piperazin-1-yl-methanone hydrochloride; and/or [2-(4-Chloro-2-ethoxy-phenyl)-4,5-bis-(4-chloro-phenyl)-4,5-dihydro-imidazol-1-yl]-(4-methyl-piperazin-1-yl)-methanone hydrochloride.

In further embodiments, the present invention relates to the use of the following specific nutlin compounds: [2-(4-Chloro-2-ethoxy-phenyl)-4,5-bis-(4-chloro-phenyl)-4,5-dihydro-imidazol-1-yl]-(4-pyrrolidin-1-yl-piperidin-1-yl)-methanone hydrochloride; [2-(4-Chloro-2-ethoxy-phenyl)-4,5-bis-(4-chloro-phenyl)-4,5-dihydro-imidazol-1-yl]-morpholin-4-yl-methanone; 1-{4-[2-(4-Chloro-2-ethoxy-phenyl)-4,5-bis-(4-chloro-phenyl)-4,5-dihydro-imidazole-1-carbonyl]-piperazin-1-yl}-ethanone; [2-(4-Chloro-2-ethoxy-phenyl)-4,5-bis-(4-chloro-phenyl)-4,5-dihydro-imidazol-1-yl]-[4-(2-hydroxy-ethyl)-piperazin-1-yl]-methanone hydrochloride; 4-[2-(4-Chloro-2-ethoxy-phenyl)-4,5-bis-(4-chloro-phenyl)-4,5-dihydro-imidazole-1-carbonyl]-piperazin-2-one; [4,5-Bis-(4-chloro-phenyl)-2-(4-ethoxy-2-isopropoxy-phenyl)-4,5-dihydro-imidazol-1-yl]-piperazin-1-yl-methanone; [4,5-Bis-(4-chloro-phenyl)-2-(2-ethoxy-5-methoxy-phenyl)-4,5-dihydro-imidazol-1-yl]-piperazin-1-yl-methanone, trifluoroacetic acid salt; [4,5-Bis-(4-chloro-phenyl)-2-(2-ethoxy-5-methoxy-phenyl)-4,5-dihydro-imidazol-1-yl]-(4-methanesulfonyl-piperazin-1-yl)-methanone; [4,5-Bis-(4-chloro-phenyl)-2-(2-ethoxy-5-methoxy-phenyl)-4,5-dihydro-imidazol-1-yl]-[4-(2-hydroxy-ethyl)-piperazin-1-yl]-methanone hydrochloride; and/or [4,5-Bis-(4-chloro-phenyl)-2-(2,5-diethoxy-phenyl)-4,5-dihydro-imidazol-1-yl]-piperazin-1-yl-methanone hydrochloride.

In still further embodiments, the present invention relates to the use of the following specific nutlin compounds: [4,5-Bis-(4-chloro-phenyl)-2-(2,5-diethoxy-phenyl)-4,5-dihydro-imidazol-1-yl]-(4-methanesulfonyl-piperazin-1-yl)-methanone; [4,5-Bis-(4-chloro-phenyl)-2-(4-ethoxy-2-isopropoxy-phenyl)-4,5-dihydro-imidazol-yl]-[4-(2-hydroxy-ethyl)-piperazin-1-yl]-methanone; 4-[4,5-Bis-(4-chloro-phenyl)-2-(4-ethoxy-2-isopropoxy-phenyl)-4,5-dihydro-imidazole-1-carbonyl]-piperazin-2-one; [4,5-Bis-(4-chloro-phenyl)-2-(5-ethoxy-2-isopropoxy-phenyl)-4,5-dihydro-imidazol-1-yl]-piperazin-1-yl-methanone; [4,5-Bis-(4-chloro-phenyl)-2-(5-ethoxy-2-isopropoxy-phenyl)-4,5-dihydro-imidazol-1-yl]-(4-methanesulfonyl-piperazin-1-yl)-methanone; [4,5-Bis-(4-chloro-phenyl)-2-(5-ethoxy-2-isopropoxy-phenyl)-4,5-dihydro-imidazol-1-yl]-[4-(2-hydroxy-ethyl)-piperazin-1-yl]-methanone hydrochloride; 1-{4-[4,5-Bis-(4-chloro-phenyl)-2-(4-ethoxy-2-isopropoxy-phenyl)-4,5-dihydro-imidazole-1-carbonyl]-piperazin-1-yl}-ethanone; 1-{4-[4,5-Bis-(4-chloro-phenyl)-2-(5-ethoxy-2-isopropoxy-phenyl)-4,5-dihydro-imidazole-1-carbonyl]-piperazin-1-yl}-ethanone; [4,5-Bis-(4-chloro-phenyl)-2-(4-ethoxy-2-isopropoxy-phenyl)-4,5-dihydro-imidazol-1-yl]-(4-pyrrolidin-1-yl-piperidin-1-yl)-methanone; and/or [4,5-Bis-(4-chloro-phenyl)-2-(2-isopropoxy-5-methoxy-phenyl)-4,5-dihydro-imidazol-1-yl]-piperazin-1-yl-methanone, trifluoroacetic acid salt.

In still further embodiments, the present invention relates to the use of the following specific nutlin compounds: The compound selected from claim 1, selected from 4,5-Bis-(4-chloro-phenyl)-2-(2,4-diisopropoxy-phenyl)4,5-dihydro-imidazol-1-yl]-piperazin-1-yl-methanone; [4,5-Bis-(4-chloro-phenyl)-2-(2,5-diisopropoxy-phenyl)-4,5-dihydro-imidazol-1-yl]-piperazin-1-yl-methanone hydrochloride; 1-[4,5-Bis-(4-chloro-phenyl)-2-(2-methoxy-5-morpholin-4-yl-methyl-phenyl)-4,5-dihydro-imidazol-1-yl]-2-methyl-propan-1-one; 1-[4,5-Bis-(4-chloro-phenyl)-2-(3-hydroxymethyl-5-methoxy-phenyl)-4,5-dihydro-imidazol-1-yl]-methyl-propan-1-one; 1-[4,5-Bis-(4-chloro-phenyl)-2-(3-hydroxymethyl-5-methoxymethyl-phenyl)-4,5-dihydro-imidazol-1-yl]-ethanone; 1-[4,5-Bis-(4-chloro-phenyl)-2-(3-methoxy-5-methoxymethyl-phenyl)-4,5-dihydro-imidazol-1-yl]-2-methyl-propan-1-one; 3-[4,5-Bis-(4-chloro-phenyl)-1-isobutyryl-4,5-dihydro-1H-imidazol-2-yl]-5-methoxymethyl-benzoic acid; 1-[4,5-Bis-(4-chloro-phenyl)-2-(5-ethoxymethyl-2,4-dimethoxy-phenyl)-4,5-dihydro-imidazol-1-yl]-2-methyl-propan-1-one; [4,5-Bis-(4-chloro-phenyl)-2-(2-ethoxy-6-methoxy-phenyl)-4,5-dihydro-imidazol-1-yl]-piperazin-1-yl-methanone; and [4,5-bis-(4-chloro-phenyl)-2-(5-ethoxymethyl-2,4-dimethoxy-phenyl)-4,5-dihydro-imidazol-1-yl]-piperazin-1-yl-methanone.

In other embodiments, the present invention relates to the use of the following specific nutlin compounds: [4,5-Bis-(4-bromo-phenyl)-2-(2-methoxy-phenyl)-4,5-dihydro-imidazol-1-yl]-[4-(2-hydroxy-ethyl)-piperazin-1-yl]-methanone; 1-[5-(4-Chloro-phenyl)-2-(4-methoxy-phenyl)-4-(4-nitro-phenyl)-4,5-dihydro-imidazol-1-yl]-2-methyl-propan-1-one; 1-[4-(4-chloro-phenyl)-2-(4-methoxy-phenyl)-5-(4-nitro-phenyl)-4,5-dihydro-imidazol-1-yl]-2-methyl-propan-1-one; 1-[4,5-Bis-(4-chloro-phenyl)-2-(2-isopropoxy-phenyl)-4,5-dihydro-imidazol-1-yl]-2-methyl-propan-1-one; [4,5-Bis-(4-chloro-phenyl)-2-(2-isopropoxy-phenyl)-4,5-dihydro-imidazol-1-yl]-(4-pyrrolidin-1-yl-piperidin-1-yl)-methanone; [4,5-Bis-(4-chloro-phenyl)-2-(2-isopropoxy-phenyl)-4,5-dihydro-imidazol-1-yl]-piperazin-1-yl-methanone; [4,5-Bis-(4-chloro-phenyl)-2-(2-isopropoxy-phenyl)-4,5-dihydro-imidazol-1-yl]-(4-dimethylamino-piperidin-1-yl)-methanone; [1,4′]Bipiperidinyl-1′-yl-[4,5-bis-(4-chloro-phenyl)-2-(2-isopropoxy-phenyl)-4,5-dihydro-imidazol-1-yl]-methanone; [4,5-Bis-(4-chloro-phenyl)-2-(2-isopropoxy-phenyl)-4,5-dihydro-imidazol-1-yl]-[4-(2-hydroxy-ethyl)-piperazin-1-yl]-methanone; and/or {(4,5-Bis-(4-chloro-phenyl)-2-[2-(2-methyl-butoxy)-phenyl]-4,5-dihydro-imidazol-1-yl}-piperazin-1-yl-methanone.

In further embodiments, the present invention relates to the use of the following specific nutlin compounds: [4,5-Bis-(4-chloro-phenyl)-2-(2-pentyloxy-phenyl)-4,5-dihydro-imidazol-1-yl]-piperazin-1-yl-methanone; [4,5-Bis-(4-chloro-phenyl)-2-(3-ethoxy-phenyl)-4,5-dihydro-imidazol-1-yl]-piperazin-1-yl-methanone, trifluoroacetic acid salt; [4,5-Bis-(4-chloro-phenyl)-2-(3-isopropoxy-phenyl)-4,5-dihydro-imidazol-1-yl]-piperazin-1-yl-methanone, trifluoroacetic acid salt; 1-{4-[4,5-Bis-(4-bromo-phenyl)-2-(2-ethoxy-phenyl)-4,5-dihydro-imidazole-1-carbonyl]-piperazin-1-yl}-ethanone; [4,5-Bis-(4-bromo-phenyl)-2-(2-ethoxy-phenyl)-4,5-dihydro-imidazol-1-yl]-[4-(2-hydroxy-ethyl)-piperazin-1-yl]-methanone; 1-{4-[4,5-Bis-(4-bromo-phenyl)-2-(2-isopropoxy-phenyl)-4,5-dihydro-imidazole-1-carbonyl]-piperazin-1-yl}-ethanone; and/or [4,5-Bis-(4-bromo-phenyl)-2-(2-isopropoxy-phenyl)-4,5-dihydro-imidazol-1-yl]-[4-(2-hydroxy-ethyl)-piperazin-1-yl]-methanone.

In certain embodiments, nutlin compounds useful according to the present invention have the following structure, with a thiophene substituent on the imidazoline ring:

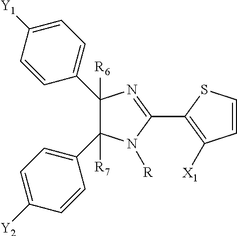

wherein substituents are as described above.

In certain embodiments, chiral nutlin compounds are useful according to the present invention, which may include, but are not limited to, those compounds disclosed in U.S. Patent Application Publication No. 2009/0143364, U.S. Patent Application Publication No. 2009/0111789, and U.S. Patent Application Publication No. 2005/02888287, each publication incorporated herein by reference in its entirety.

In some embodiments, nutlin compounds according to the present invention include, but are not limited to, imidazopyridinone compounds such as those disclosed in U.S. Pat. No. 7,625,895, incorporated herein by reference in its entirety. In certain embodiments, imidazopyridinone compounds according to the following structure are useful according to the present invention:

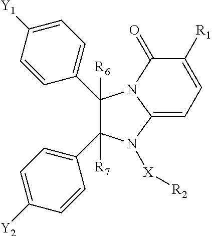

wherein:

Y₁ and Y₂ are independently selected from the group consisting of halogen, trifluoromethyl, —NO₂, —C≡N, and —C≡CH;

X is selected from the group consisting of —SO₂, —C═O and —C═OCH₂;

R₁ is selected from the group consisting of hydrogen, halogen, aryl, substituted aryl, heterocycle, substituted heterocycle, alkenyl and C═OR₃;

R₂ is selected from the group consisting of substituted or unsubstituted cycloalkyl, aryl, heteroaryl and heterocycle;

R₃ is alkoxy, amino, cycloamino, heterocycle or substituted heterocycle;

and pharmaceutically acceptable salts and esters thereof.

In some embodiments, imidazopyridinone compounds useful according to the present invention include, but are not limited to, 5-[rac-cis-2,3-Bis-(4-chloro-phenyl)-5-oxo-2,3-dihydro-5H-imidazo[1,2-a]pyridine-1-sulfonyl]-2-fluoro-benzonitrile; 3-2R*,3S*-Bis-(4-chloro-phenyl)-5-oxo-2,3-dihydro-5H-imidazo[1,2-a]pyridine-1-sulfonyl]-benzonitrile; 5-[2R*,3 S*-Bis-(4-chloro-phenyl)-5-oxo-2,3-dihydro-5H-imidazo[1,2-a]pyridine-1-sulfonyl]-2-fluoro-benzonitrile; 2R*,3S*-Bis-(4-chloro-phenyl)-1-(2-fluoro-benzenesulfonyl)-2,3-dihydro-1H-imidazo[1,2-a]pyridin-5-one; rac-3-[cis-2,3-Bis-(4-chloro-phenyl)-5-oxo-2,3-dihydro-5H-imidazo[1,2-a]pyridine-1-sulfonyl]-benzonitrile; rac4-[-cis-2,3-Bis-(4-chloro-phenyl)-5-oxo-2,3-dihydro-5H-imidazo[1,2-a]pyridine-1-sulfonyl]-benzonitrile; rac-cis-2,3-Bis-(4-chloro-phenyl)-1-(2-fluoro-benzenesulfonyl)-2,3-dihydro-1H-imidazo[1,2-a]pyridin-5-one; rac-cis-2,3-Bis-(4-chloro-phenyl)-1-(2,4-difluoro-benzenesulfonyl)-2,3-dihydro-1H-imidazo[1,2-a]pyridin-5-one; rac-cis-2,3-Bis-(4-chloro-phenyl)-1-(2,5-dimethoxy-benzenesulfonyl)-2,3-dihydro-1H-imidazo[1,2-a]pyridin-5-one; rac-cis-1-(2-Chloro-benzoyl)-2,3-bis-(4-chloro-phenyl)-2,3-dihydro-1H-imidazo[1,2-a]pyridin-5-one; rac-cis-1-(2-Chloro-benzenesulfonyl)-2,3-bis-(4-chloro-phenyl)-2,3-dihydro-1H-imidazo[1,2-a]pyridin-5-one; rac-cis-2,3-Bis-(4-chloro-phenyl)-1-(thiophene-3-sulfonyl)-2,3-dihydro-1H-imidazo[1,2-a]pyridin-5-one; rac-cis-2,3-Bis-(4-chloro-phenyl)-1-(1-methyl-1H-imidazole-4-sulfonyl)-2,3-dihydro-1H-imidazo[1,2-a]pyridin-5-one; rac-cis-2,3-Bis-(4-chloro-phenyl)-1-(4-fluoro-benzenesulfonyl)-2,3-dihydro-1H-imidazo[1,2-a]pyridin-5-one; rac-cis-1-Benzenesulfonyl-rac-cis-bis-(4-chloro-phenyl)-2,3-dihydro-1H-imidazo[1,2-a]pyridin-5-one; rac-cis-2,3-Bis-(4-chloro-phenyl)-1-(2,6-difluoro-benzenesulfonyl)-2,3-dihydro-1H-imidazo[1,2-a]pyridin-5-one; rac-cis-2,3-Bis-(4-chloro-phenyl)-1-(thiophene-2-sulfonyl)-2,3-dihydro-1H-imidazo[1,2-a]pyridin-5-one; rac-cis-1-(3-Chloro-2-fluoro-benzenesulfonyl)-2,3-bis-(4-chloro-phenyl)-2,3-dihydro-1H-imidazo[1,2-a]pyridin-5-one; rac-cis-2,3-Bis-(4-chloro-phenyl)-1-(2-fluoro-5-methyl-benzenesulfonyl)-2,3-dihydro-1H-imidazo[1,2-a]pyridin-5-one and rac-1-(2-Chloro-4-fluoro-benzenesulfonyl)-cis-2,3-bis-(4-chloro-phenyl)-2,3-dihydro-1H-imidazo[1,2-a]pyridin-5-one.

In other embodiments, imidazopyridinone compounds useful according to the present invention include, but are not limited to, rac-cis-2,3-Bis-(4-chloro-phenyl)-1-(4-methoxy-benzenesulfonyl)-2,3-dihydro-1H-imidazo[1,2-a]pyridin-5-one; rac-cis-2,3-Bis-(4-chloro-phenyl)-1-(toluene-3-sulfonyl)-2,3-dihydro-1H-imidazo[1,2-a]pyridin-5-one; rac-cis-2,3-Bis-(4-chloro-phenyl)-1-(3-methoxy-benzenesulfonyl)-2,3-dihydro-1H-imidazo[1,2-a]pyridin-5-one; rac-cis-1-(5-Chloro-2-fluoro-benzenesulfonyl)-2,3-bis-(4-chloro-phenyl)-2,3-dihydro-1H-imidazo[1,2-a]pyridin-5-one; rac-cis-1-(4-Chloro-2-fluoro-benzenesulfonyl)-2,3-bis-(4-chloro-phenyl)-2,3-dihydro-1H-imidazo[1,2-a]pyridin-5-one; rac-cis-2,3-Bis-(4-chloro-phenyl)-1-(3,4-difluoro-benzenesulfonyl)-2,3-dihydro-1H-imidazo[1,2-a]pyridin-5-one; rac-3-[cis-2,3-Bis-(4-chloro-phenyl)-5-oxo-2,3-dihydro-5H-imidazo[1,2-a]pyridine-1-sulfonyl]-benzoic acid methyl ester; rac-cis-1-(5-Chloro-2-methoxy-benzenesulfonyl)-2,3-bis-(4-chloro-phenyl)-2,3-dihydro-1H-imidazo1,2-a]pyridin-5-one; rac-cis-1-(3-Chloro-benzenesulfonyl)-2,3-bis-(4-chloro-phenyl)-2,3-dihydro-1H-imidazo[1,2-a]pyridin-5-one; rac-cis-2,3-Bis-(4-chloro-phenyl)-1-(3-fluoro-benzenesulfonyl)-2,3-dihydro-1H-imidazo[1,2-a]pyridin-5-one; rac-cis-2,3-Bis-(4-chloro-phenyl)-1-(toluene-2-sulfonyl)-2,3-dihydro-1H-imidazo[1,2-a]pyridin-5-one; rac-cis-1-(4-Chloro-benzenesulfonyl)-2,3-bis-(4-chloro-phenyl)-2,3-dihydro-1H-imidazo[1,2-a]pyridin-5-one; rac-cis-2,3-Bis-(4-chloro-phenyl)-1-(2-fluoro-benzoyl)-2,3-dihydro-1H-imidazo[1,2-a]pyridin-5-one; rac-cis-1-(3-Chloro-4-fluoro-benzenesulfonyl)-2,3-bis-(4-chloro-phenyl)-2,3-dihydro-1H-imidazo[1,2-a]pyridin-5-one; rac-cis-2,3-Bis-(4-chloro-phenyl)-1-(4-fluoro-2-methyl-benzenesulfonyl)-2,3-dihydro-1H-imidazo[1,2-a]pyridin-5-one; rac-cis-2,3-Bis-(4-chloro-phenyl)-1-(5-fluoro-2-methyl-benzenesulfonyl)-2,3-dihydro-1H-imidazo[1,2-a]pyridin-5-one; rac-cis-2,3-Bis-(4-chloro-phenyl)-1-(3-methoxy-benzoyl)-2,3-dihydro-1H-imidazo[1,2-a]pyridin-5-one; rac-cis-2,3-Bis-(4-chloro-phenyl)-1-(2-trifluoromethoxy-benzenesulfonyl)-2,3-dihydro-1H-imidazo[1,2-a]pyridin-5-one; rac-cis-2,3-Bis-(4-chloro-phenyl)-1-isobutyryl-2,3-dihydro-1H-imidazo[1,2-a]pyridin-5-one and 2R*,3S*-Bis-(4-chloro-phenyl)-1-(2-fluoro-benzenesulfonyl)-2,3-dihydro-1H-imidazo[1,2-a]pyridin-5-one.

In further embodiments, imidazopyridinone compounds useful according to the present invention include, but are not limited to, rac-cis-2,3-Bis-(4-chloro-phenyl)-1-cyclopropanesulfonyl-2,3-dihydro-1H-imidazo[1,2-a]pyridin-5-one; rac-cis-1-(3-Chloro-benzoyl)-2,3-bis-(4-chloro-phenyl)-2,3-dihydro-1H-imidazo[1,2-a]pyridin-5-one; rac-cis-2,3-Bis-(4-chloro-phenyl)-1-(3-trifluoromethoxy-benzenesulfonyl)-2,3-dihydro-1H-imidazo[1,2-a]pyridin-5-one; rac-cis-2,3-Bis-(4-chloro-phenyl)-1-(3-fluoro-benzoyl)-2,3-dihydro-1H-imidazo[1,2-a]pyridin-5-one; rac-cis-2,3-Bis-(4-chloro-phenyl)-1-[2-(2,5-dimethoxy-phenyl)-acetyl]-2,3-dihydro-1H-imidazo[1,2-a]pyridin-5-one; rac-cis-2,3-Bis-(4-chloro-phenyl)-1-(furan-2-carbonyl)-2,3-dihydro-1H-imidazo[1,2-a]pyridin-5-one; rac-cis-2,3-Bis-(4-chloro-phenyl)-1-(2-methoxy-benzoyl)-2,3-dihydro-1H-imidazo[1,2-a]pyridin-5-one; 1-(2-Chloro-benzoyl)2R*,3S*-bis-(4-chloro-phenyl)-2,3-dihydro-1H-imidazo[1,2-a]pyridin-5-one; rac-cis-2,3-Bis-(4-chloro-phenyl)-1-cyclopentanecarbonyl-2,3-dihydro-1H-imidazo[1,2-a]pyridin-5-one; rac-cis-1-(3-Chloro-2-methyl-benzenesulfonyl)-2,3-bis-(4-chloro-phenyl)-2,3-dihydro-1H-imidazo[1,2-a]pyridin-5-one; rac-cis-2,3-Bis-(4-chloro-phenyl)-1-(2-fluoro-benzenesulfonyl)-2,3-dihydro-6-iodo-1H-imidazo[1,2-a]pyridin-5-one; rac-cis-6-(4-Acetyl-piperazine-1-carbonyl)-2,3-bis-(4-chloro-phenyl)-1-(2-fluoro-benzenesulfonyl)-2,3-dihydro-1H-imidazo[1,2-a]pyridin-5-one; -6-(4-Acetyl-piperazine-1-carbonyl)-2R*,3S*-bis-(4-chloro-phenyl)-1-(2-fluoro-benzenesulfonyl)-2,3-dihydro-1H-imidazo[1,2-a]pyridin-5-one; rac-cis-2,3-Bis-(4-chloro-phenyl)-1-(2-fluoro-benzenesulfonyl)-6-[4-(2-morpholin-4-yl-2-oxo-ethyl)-piperazine-1-carbonyl]-2,3-dihydro-1H-imidazo[1,2-a]pyridin-5-one; rac-cis-2,3-Bis-(4-chloro-phenyl)-1-(2-fluoro-benzenesulfonyl)-6-(morpholine-4-carbonyl)-2,3-dihydro-1H-imidazo[1,2-a]pyridin-5-one; N-(2-{4-[rac-cis-2,3-Bis-(4-chloro-phenyl)-1-(2-fluoro-benzenesulfonyl)-5-oxo-1,2,3,5-tetrahydro-imidazo[1,2-a]pyridine-6-carbonyl]-piperazin-1-yl}-ethyl)-methanesulfonamide; rac-cis-2,3-Bis-(4-chloro-phenyl)-1-(2-fluoro-benzenesulfonyl)-6-[4-(2-morpholin-4-yl-ethyl)-piperazine-1-carbonyl]-2,3-dihydro-1H-imidazo[1,2-a]pyridin-5-one; rac-cis-2,3-Bis-(4-chloro-phenyl)-1-(2-fluoro-benzenesulfonyl)-6-[4-(3-methanesulfonyl-propyl)-piperazine-1-carbonyl]-2,3-dihydro-1H-imidazo[1,2-a]pyridin-5-one; rac-cis-2,3-Bis-(4-chloro-phenyl)-1-(2-fluoro-benzenesulfonyl)-6-(4-methyl-piperazine-1-carbonyl)-2,3-dihydro-1H-imidazo[1,2-a]pyridin-5-one and rac-cis-2,3-Bis-(4-chloro-phenyl)-6-(4-ethanesulfonyl-piperazine-1-carbonyl)-1-(2-fluoro-benzenesulfonyl)-2,3-dihydro-1H-imidazo[1,2-a]pyridin-5-one.

In still further, imidazopyridinone compounds useful according to the present invention include, but are not limited to, 1-[rac-cis-2,3-Bis-(4-chloro-phenyl)-1-(2-fluoro-benzenesulfonyl)-5-oxo-1,2,3,5-tetrahydro-imidazo[1,2-a]pyridin-6-yl]-2-[4-(3-methanesulfonyl-propyl)-piperazin-1-yl]-ethane-1,2-dione; rac-cis-2,3-Bis-(4-chloro-phenyl)-1-(2-fluoro-benzenesulfonyl)-5-oxo-1,2,3,5-tetrahydro-imidazo[1,2-a]pyridine-6-carboxylic acid methyl ester; rac-cis-2,3-Bis-(4-chloro-phenyl)-1-(2-fluoro-benzenesulfonyl)-5-oxo-1,2,3,5-tetrahydro-imidazo[1,2-a]pyridine-6-carboxylic acid methylamide; 6-(4-Acetyl-piperazine-1-carbonyl)2R*,3S*-bis-(4-chloro-phenyl)-1-(2-fluoro-benzenesulfonyl)-2,3-dihydro-1H-imidazo[1,2-a]pyridin-5-one; rac-3-[cis-2,3-Bis-(4-chloro-phenyl)-6-(morpholine-4-carbonyl)-5-oxo-2,3-dihydro-5H-imidazo[1,2-a]pyridine-1-sulfonyl]-benzoic acid methyl ester; rac-3-{cis-2,3-Bis-(4-chloro-phenyl)-6-[4-(2-morpholin-4-yl-2-oxo-ethyl)-piperazine-1-carbonyl]-5-oxo-2,3-dihydro-5H-imidazo[1,2-a]pyridine-1-sulfonyl}-benzoic acid methyl ester; rac-3-[cis-2,3-Bis-(4-chloro-phenyl)-6-(4-methyl-piperazine-1-carbonyl)-5-oxo-2,3-dihydro-5H-imidazo[1,2-a]pyridine-1-sulfonyl]-benzoic acid methyl ester; rac-3-[cis-6-(4-Acetyl-piperazine-1-carbonyl)-2,3-bis-(4-chloro-phenyl)-5-oxo-2,3-dihydro-5H-imidazo[1,2-a]pyridine-1-sulfonyl]-benzoic acid methyl ester; rac-3-[cis-2,3-Bis-(4-chloro-phenyl)-6-(4-ethanesulfonyl-piperazine-1-carbonyl)-5-oxo-2,3-dihydro-5H-imidazo[1,2-a]pyridine-1-sulfonyl]-benzoic acid methyl ester; rac-3-{cis-2,3-Bis-(4-chloro-phenyl)-6-[4-(2-imidazol-1-yl-ethyl)-piperazine-1-carbonyl]-5-oxo-2,3-dihydro-5H-imidazo[1,2-a]pyridine-1-sulfonyl}-benzoic acid methyl ester; 3-{(2R,3S)-2,3-Bis-(4-chloro-phenyl)-6-[4-(2-morpholin-4-yl-2-oxo-ethyl)-piperazin-1-ylmethyl]-5-oxo-2,3-dihydro-5H-imidazo[1,2-a]pyridine-1-sulfonyl}-benzonitrile; rac-3-[cis-2,3-Bis-(4-chloro-phenyl)-6-(morpholine-4-carbonyl)-5-oxo-2,3-dihydro-5H-imidazo[1,2-a]pyridine-1-sulfonyl]-benzonitrile; rac-3-[(6-(4-Acetyl-piperazine-1-carbonyl)-cis-2,3-bis-(4-chloro-phenyl)-5-oxo-2,3-dihydro-5H-imidazo[1,2-a]pyridine-1-sulfonyl]-benzonitrile; rac-3-{cis-2,3-Bis-(4-chloro-phenyl)-5-oxo-6-[4-(3,3,3-trifluoro-propionyl)-piperazine-1-carbonyl]-2,3-dihydro-5H-imidazo[1,2-a]pyridine-1-sulfonyl}-benzonitrile; 3-[2R*,3S*-Bis-(4-chloro-phenyl)-6-(morpholine-4-carbonyl)-5-oxo-2,3-dihydro-5H-imidazo[1,2-a]pyridine-1-sulfonyl]-benzonitrile; 3-[2R*,3 S*-Bis-(4-chloro-phenyl)-6-(morpholine-4-carbonyl)-5-oxo-2,3-dihydro-5H-imidazo[1,2-a]pyridine-1-sulfonyl]-benzonitrile; rac-cis-1-Acetyl-2,3-bis-(4-chloro-phenyl)-6-(3,4-dimethoxy-phenyl)-2,3-dihydro-1H-imidazo[1,2-a]pyridin-5-one; rac-cis-1-Acetyl-2,3-bis-(4-chloro-phenyl)-6-(4-methanesulfonyl-phenyl)-2,3-dihydro-1H-imidazo[1,2-a]pyridin-5-one; rac-cis-1-Acetyl-2,3-bis-(4-chloro-phenyl)-6-(3-methanesulfonyl-phenyl)-2,3-dihydro-1H-imidazo[1,2-a]pyridin-5-one; rac-cis-1-Acetyl-6-(1-benzyl-1H-pyrazol-4-yl)-2,3-bis-(4-chloro-phenyl)-2,3-dihydro-1H-imidazo[1,2-a]pyridin-5-one; rac-cis-2,3-Bis-(4-chloro-phenyl)-1-(2-fluoro-benzenesulfonyl)-6-(3-methanesulfonyl-phenyl)-2,3-dihydro-1H-imidazo[1,2-a]pyridin-5-one; rac-cis-2,3-Bis-(4-chloro-phenyl)-1-(2-fluoro-benzenesulfonyl)-6-(2-methyl-propenyl)-2,3-dihydro-1H-imidazo[1,2-a]pyridin-5-one; rac-3-[cis-2,3-Bis-(4-chloro-phenyl)-6-morpholin-4-ylmethyl-5-oxo-2,3-dihydro-5H-imidazo[1,2-a]pyridine-1-sulfonyl]-benzonitrile; rac-3-[cis-6-(4-Acetyl-piperazin-1-ylmethyl)-2,3-bis-(4-chloro-phenyl)-5-oxo-2,3-dihydro-5H-imidazo[1,2-a]pyridine-1-sulfonyl]-benzonitrile and rac-3-[cis-2,3-Bis-(4-chloro-phenyl)-5-oxo-2,3-dihydro-5H-imidazo[1,2-a]pyridine-1-sulfonyl]-benzoic acid.

In certain embodiments, the invention relates to the use of spirooxindole-based MDM2 inhibitors. For example, in some embodiments, spirooxindoles such as those disclosed in Shangary S. et al., Proc. Natl. Acad. Sci. 105(10):3933-38 (Mar. 11, 2008), and Ding, K. et at, J. Med. Chem. 49:3432-35 (2006), both incorporated herein by reference, may be used according to the methods of the present invention.

In certain embodiments, the invention relates to the use of diazepine-based, benzodiazepine-based or benzodiazepinedione-based MDM2 inhibitors. For example, in certain embodiments, 1,4-diazepines such as those disclosed in U.S. Pat. No. 7,115,598, incorporated herein by reference, may be used according to the present invention. In some embodiments, benzodiazepines such as those disclosed in Grasberger, B. L. et al., J. Med. Chem. 48(4):909-12 (2005), incorporated herein by reference, may be used. In certain embodiments, 1,4-benzodiazepines such as those disclosed in U.S. Pat. No. 7,067,512, incorporated herein by reference, may be used according to the present invention.

In some embodiments, the invention relates to the use of sulfonamide-based MDM2 inhibitors (e.g., bisarylsulfonamides). For example, compounds such as those disclosed in United States Patent Application Publication No. 2005-0215548, incorporated herein by reference, may be used according to the present invention.

It is to be understood that this invention also covers any analogue of the compounds discussed herein. Specifically, in certain embodiments, the invention relates to analogues of the above-referenced nutlin compounds. Analogues, in this sense, refers, for example, to any similar compounds having a structure distinct from those structures set forth herein, but which exhibit the desired activity; namely, MDM2 inhibition activity.

In some embodiments of the present invention, therapeutically inactive prodrugs are provided. Prodrugs are compounds which, when administered to a mammal, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound.

A number of prodrug ligands are known. In general, alkylation, acylation, or other lipophilic modification of one or more heteroatoms of the compound, such as a free amine or carboxylic acid residue, may reduce polarity and allow for the compound's passage into cells. Examples of substituent groups that can replace one or more hydrogen atoms on a free amine and/or carboxylic acid moiety include, but are not limited to, the following: aryl; steroids; carbohydrates (including sugars); 1,2-diacylglycerol; alcohols; acyl (including lower acyl); alkyl (including lower alkyl); sulfonate ester (including alkyl or arylalkyl sulfonyl, such as methanesulfonyl and benzyl, wherein the phenyl group is optionally substituted with one or more substituents as provided in the definition of an aryl given herein); optionally substituted arylsulfonyl; lipids (including phospholipids); phosphotidylcholine; phosphocholine; amino acid residues or derivatives; amino acid acyl residues or derivatives; peptides; cholesterols; or other pharmaceutically acceptable leaving groups which, when administered in vivo, provide the free amine. Any of these moieties can be used in combination with the disclosed active agents to achieve a desired effect.

In some embodiments, compounds with one or more chiral centers are provided. While racemic mixtures of compounds of the invention may be active, selective, and bioavailable, isolated isomers may be of interest as well.

The compounds disclosed herein as active agents may contain chiral centers, which may be either of the (R) or (S) configuration, or which may comprise a mixture thereof. Accordingly, the present invention also includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds and prodrugs of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the compounds described herein.

Various methods are known in the art for preparing optically active forms and determining activity. Such methods include standard tests described herein and other similar tests which are well known in the art. Examples of methods that can be used to obtain optical isomers of the compounds according to the present invention include the following:

i) physical separation of crystals whereby macroscopic crystals of the individual enantiomers are manually separated. This technique may particularly be used when crystals of the separate enantiomers exist (i.e., the material is a conglomerate), and the crystals are visually distinct;

ii) simultaneous crystallization whereby the individual enantiomers are separately crystallized from a solution of the racemate, possible only if the latter is a conglomerate in the solid state;

iii) enzymatic resolutions whereby partial or complete separation of a racemate by virtue of differing rates of reaction for the enantiomers with an enzyme;

iv) enzymatic asymmetric synthesis, a synthetic technique whereby at least one step of the synthesis uses an enzymatic reaction to obtain an enantiomerically pure or enriched synthetic precursor of the desired enantiomer;

v) chemical asymmetric synthesis whereby the desired enantiomer is synthesized from an achiral precursor under conditions that produce asymmetry (i.e., chirality) in the product, which may be achieved using chiral catalysts or chiral auxiliaries;

vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;

viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors whereby the desired enantiomer is obtained from non-chiral starting materials and where the stereochemical integrity is not or is only minimally compromised over the course of the synthesis;

x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography whereby the racemate is volatilized and enantiomers are separated by virtue of their differing interactions in the gaseous mobile phase with a column containing a fixed non-racemic chiral adsorbent phase;

xii) extraction with chiral solvents whereby the enantiomers are separated by virtue of preferential dissolution of one enantiomer into a particular chiral solvent; and

xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The compound optionally may be provided in a composition that is enantiomerically enriched, such as a mixture of enantiomers in which one enantiomer is present in excess, in particular, to the extent of 95% or more, 96% or more, 97% or more, 98% or more, or 99% or more, including 100%.

The terms (R), (S), (R,R), (S,S), (R,S) and (S,R) as used herein mean that the composition contains a greater proportion of the named isomer of the compound in relation to other isomers. In a preferred embodiment, these terms indicate that the composition contains at least 90% by weight of the named isomer and 10% by weight or less of the one or more other isomers; or more preferably about 95% by weight of the named isomer and 5% or less of the one or more other isomers. In some embodiments, the composition may contain at least 99% by weight of the named isomer and 1% or less by weight of the one or more other isomers, or may contain 100% by weight of the named isomer and 0% by weight of the one of more other isomers. These percentages are based on the total amount of the compound of the present invention present in the composition.

The compounds of the present invention may be utilized per se or in the form of a pharmaceutically acceptable ester, amide, salt, solvate, prodrug, or isomer. For example, the compound may be provided as a pharmaceutically acceptable salt. If used, a salt of the drug compound should be both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts can be prepared by reaction of the drug with an organic or inorganic acid, using standard methods detailed in the literature.

Examples of pharmaceutically acceptable salts of the compounds useful according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzenesulfonic, and isethionic acids. Other useful acid addition salts include propionic acid, glycolic acid, oxalic acid, malic acid, malonic acid, benzoic acid, cinnamic acid, mandelic acid, salicylic acid, and the like. Particular example of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxyenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

An acid addition salt may be reconverted to the free base by treatment with a suitable base. Preparation of basic salts of acid moieties which may be present on a compound or prodrug useful according to the present invention may be prepared in a similar manner using a pharmaceutically acceptable base, such as sodium hydroxide, potassium hydroxide, ammonium hydroxide, calcium hydroxide, triethylamine, or the like.

Esters of the active agent compounds according to the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from an anhydride or an acid chloride by reaction with ammonia or a lower alkyl amine. Moreover, esters and amides of compounds of the invention can be made by reaction with a carbonylating agent (e.g., ethyl formate, acetic anhydride, methoxyacetyl chloride, benzoyl chloride, methyl isocyanate, ethyl chloroformate, methanesulfonyl chloride) and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C. Prodrugs are typically prepared by covalent attachment of a moiety, which results in a compound that is therapeutically inactive until modified by an individual's metabolic system. Examples of pharmaceutically acceptable solvates include, but are not limited to, compounds according to the invention in combination with water, isopropanol, ethanol, methanol, DMSO, ethyl acetate, acetic acid, or ethanolamine.

In the case of solid compositions, it is understood that the compounds used in the methods of the invention may exist in different forms. For example, the compounds may exist in stable and metastable crystalline forms and isotropic and amorphous forms, all of which are intended to be within the scope of the present invention.

If a compound useful as an active agent according to the invention is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such a p-toluenesulfonic acid or ethanesulfonic acid, or the like.

If a compound described herein as an active agent is an acid, the desired salt may be prepared by any suitable method known to the art, including treatment of the free acid with an inorganic or organic base, such as an amine (primary, secondary or tertiary), an alkali metal or alkaline earth metal hydroxide or the like. Illustrative examples of suitable salts include organic salts derived from amino acids such as glycine and arginine, ammonia, primary, secondary and tertiary amines, and cyclic amines such as piperidine, morpholine and piperazine, and inorganic salts derived from sodium, calcium, potassium, magnesium, manganese, iron, copper, zinc, aluminum and lithium.

### Compositions

While it is possible for the compounds disclosed in the present application to be administered in the raw chemical form, it is preferred for the compounds to be delivered as a pharmaceutical formulation. Accordingly, there are provided by the present invention pharmaceutical compositions comprising at least one compound characterized as an MDM2 inhibitor. As such, the formulations of the present invention comprise a compound of any of the classes noted herein, as described above, or a pharmaceutically acceptable ester, amide, salt, or solvate thereof, together with one or more pharmaceutically acceptable carriers therefor, and optionally, other therapeutic ingredients.

By “pharmaceutically acceptable carrier” is intended a carrier, adjuvant, accessory, or excipient that is conventionally used in the art to facilitate the storage, administration, and/or the healing effect of the agent. The carrier(s) must be pharmaceutically acceptable in the sense of being compatible with the other ingredients of the formulation and not unduly deleterious to the recipient thereof. A carrier may also reduce any undesirable side effects of the agent. Such carriers are known in the art. See, Wang et al. (1980) J. Parent. Drug Assn. 34(6):452-462, herein incorporated by reference in its entirety.

Adjuvants or accessory ingredients for use in the formulations of the present invention can include any pharmaceutical ingredient commonly deemed acceptable in the art, such as fillers, stabilizers, diluents, buffers, binders, disintegrants, thickeners, lubricants, preservatives (including antioxidants), flavoring and coloring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Exemplary excipients include water, saline, dextrose, glycerol, ethanol, and combinations thereof. Other exemplary pharmaceutical excipients and/or additives suitable for use in the compositions according to the invention are listed in Remington: The Science & Practice of Pharmacy, 21^{st} ed., Lippincott Williams & Wilkins (2006); in the Physician's Desk Reference, 64^{th} ed., Thomson PDR (2010); and in Handbook of Pharmaceutical Excipients, 6^{th} ed., Eds. Raymond C. Rowe et al., Pharmaceutical Press (2009), which are incorporated herein by reference.

In certain embodiments, the formulation is designed for ocular delivery, and carriers for such purposes are described, for example, in Glenn J. Jaffe et al., Eds., Intraocular Drug Delivery (2006), incorporated herein by reference in its entirety. In general, ocular formulations comprise one or more active compounds and various ophthalmologically acceptable excipients in the form of, for example, a solution, an ointment, or a suspension. The one or more carriers may comprise any substances that are non-irritating to the eye, permit diffusion of the drug into the ocular fluid, and allow for the activity of the medicament for a reasonable period of time under storage conditions. Particularly preferred carriers for ocular delivery according to the present invention include sterile isotonic solutions such as isotonic sodium chloride or boric acid solutions. Such carriers typically comprise sodium chloride and/or boric acid, as well as sterile distilled or purified water. In some formulations, they may comprise phosphate buffered saline (PBS). Carriers may also comprise dimethylsulfoxide (DMSO). Other preferred carriers for ocular delivery comprise white petrolatum, mineral oil and/or polyethylene-mineral oil gel.

An excipient is ophthalmologically acceptable if it is non-irritating to the eye. In some embodiments, excipients can include, for example, a tonicifier, a preservative, a surfactant, a buffering system, a chelating agent, and/or a viscosity-modifying agent (e.g., methylcellulose) as well as other stabilizing agents. Preferably, the pH of a formulation for ocular delivery is in the range of 5-8, and more preferably close to the pH of tears (i.e., 7.4). Accordingly, pH adjusting agents may be included in the formulations; for example, the formulations may comprise such agents as sodium hydroxide, hydrochloric acid, and/or sulfuric acid. In certain preferred embodiments, one or more preservatives are included in formulations according to the present invention. In preferred embodiments, such preservatives include, but are not limited to, benzalkonium chloride, parabens, organic mercurial compounds, sorbic acid, EDTA, benzylchromium chloride, and/or chlorobutanol. In some embodiments, the ocular formulations further comprise such excipients as phenylmercuric nitrate, sodium sulfate, sodium sulfite, sodium phosphate, and/or monosodium phosphate. In some embodiments, the compositions include mucomimetics such as hyaluronic acid, chondroitin sulfate, hydroxypropyl methylcellulose, and/or or poly(vinyl alcohol).

Depending on the method of administration, the formulation may be provided in any form. For example, in some embodiments according to the present invention, the formulation may comprise a solution or a suspension. In some embodiments, the formulation may comprise an ointment or gel. In some embodiments, the formulation may comprise a drug delivery device. For example, in certain embodiments, the formulation may comprise an ocular delivery device, such as a drug-impregnated solid carrier that is inserted into the eye. In some embodiments, the formulation may comprise a tablet or capsule.

Where the formulation comprises a tablet, binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluoses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the formulation according to the present invention may be anionic, cationic, amphoteric, or nonionic surface active agents. Stabilizers may be included in the formulations to inhibit or lessen reactions leading to decomposition of the active agent, such as oxidative reactions.

Formulations of the present invention may include short-term, rapid-onset, rapid-offset, controlled release, sustained release, delayed release, and pulsatile release formulations, providing the formulations achieve administration of a compound as described herein. See Remington's Pharmaceutical Sciences (18^{th} ed.; Mack Publishing Company, Eaton, Pa., 1990), herein incorporated by reference in its entirety.

Pharmaceutical formulations according to the present invention are suitable for various modes of delivery, including oral, parenteral (including intravenous, intramuscular, subcutaneous, intradermal, and transdermal), topical (including dermal, buccal, and sublingual), and rectal administration. The most useful and/or beneficial mode of administration can vary, especially depending upon the condition of the recipient and the disorder being treated and/or prevented. In certain embodiments, the formulations according to the present invention can be formulated for direct delivery to the eye, including, but not limited to, by intraocular injection, by direct injection into a given compartment of the eye, such as the vitreous, the cornea, or the retina, by application of a patch on the eye, by direct application of an ointment, spray, or droppable liquid to the eye. In some preferred embodiments, the formulation is for intravitreal, subconjunctival, or periocular delivery, or is formulated for topical delivery to the eye. In some embodiments, an intraocular implant may be used to deliver a MDM2 inhibitor according to the present invention. In certain embodiments, such implants can be biodegradable and/or biocompatible implants. The implants may be inserted into a chamber of the eye, such as the anterior or posterior chambers, or may be implanted in the sclera, transchoroidal space, or an avascularized region exterior to the vitreous. In one embodiment, the implant may be positioned over an avascular region, such as on the sclera, so as to allow for transscleral diffusion of the drug to the desired site of treatment.

The pharmaceutical formulations may be conveniently made available in a unit dosage form, whereby such formulations may be prepared by any of the methods generally known in the pharmaceutical arts. Generally speaking, such methods of preparation comprise combining (by various methods) an active agent, such as an MDM2 inhibitor (or a pharmaceutically acceptable ester, amide, salt, or solvate thereof), with a suitable carrier or other adjuvant, which may consist of one or more ingredients. The combination of the active ingredient with the one or more adjuvants is then physically treated to present the formulation in a suitable form for delivery (e.g., shaping into a tablet or forming an aqueous suspension).

Pharmaceutical formulations according to the present invention suitable as oral dosage may take various forms, such as tablets, capsules, caplets, and wafers (including rapidly dissolving or effervescing), each containing a predetermined amount of the active agent. The formulations may also be in the form of a powder or granules, a solution or suspension in an aqueous or non-aqueous liquid, and as a liquid emulsion (oil-in-water and water-in-oil). The active agent may also be delivered as a bolus, electuary, or paste. It is generally understood that methods of preparations of the above dosage forms are generally known in the art, and any such method would be suitable for the preparation of the respective dosage forms for use in delivery of the compounds according to the present invention. Solid formulations of the invention, when particulate, will typically comprise particles with sizes ranging from about 1 nanometer to about 500 microns. In general, for solid formulations intended for intravenous administration, particles will typically range from about 1 nm to about 10 microns in diameter.

A tablet containing a compound according to the present invention may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agent.

Solid dosage forms may be formulated so as to provide a delayed release of the active agent, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agent over a prolonged period of time), and may or may not also be delayed release. Sustained release formulations are known in the art and are generally prepared by dispersing a drug within a matrix of a gradually degradable or hydrolyzable material, such as an insoluble plastic, a hydrophilic polymer, or a fatty compound. For example, the drug may be contained within nanoparticles. Alternatively, a solid dosage form may be coated with such a material.

Formulations for parenteral administration include aqueous and non-aqueous sterile injection solutions, which may further contain additional agents, such as anti-oxidants, buffers, bacteriostats, and solutes, which render the formulations isotonic with the blood of the intended recipient. The formulations may include aqueous and non-aqueous sterile suspensions, which contain suspending agents and thickening agents. Such formulations for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compounds according to the present invention may also be administered transdermally, wherein the active agent is incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agent is contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Formulations for rectal delivery of the compounds of the present invention include rectal suppositories, creams, ointments, and liquids. Suppositories may be presented as the active agent in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Formulations for ocular delivery may be provided, for example, as a solution, suspension, ointment, gel, or as an ocular delivery device. In embodiments wherein the formulation is a suspension, the particle sizes therein should be less than 10 μm to minimize eye irritation. In embodiments wherein the formulation is a solution or suspension, the amount delivered to the patient should be selected so as to avoid excessive spillage from the eye. For example, in certain embodiments, the amount delivered should not exceed 75 preferably 50 μl or less, to avoid excessive spillage from the eye. In embodiments wherein the formulation is an ocular delivery device, the carrier may comprise any one of a variety of polymers. Drug release may occur from the delivery device via dissolution of the device and/or osmosis of the active agent from the device.

The amount of the MDM2 inhibitor contained in the formulation will vary depending on the specific compound or prodrug selected, dosage form, target patient population, and other considerations, and will be readily determined by one skilled in the art. The amount of the compound in the formulation will be that amount necessary to deliver a therapeutically effective amount of the compound to a patient in need thereof to achieve at least one of the therapeutic effects associated with the compounds of the invention. In practice, this will vary widely depending upon the particular compound, its activity, the severity of the condition to be treated, the patient population, the stability of the formulation, and the like. Compositions will generally contain anywhere from about 1% by weight to about 99% by weight of a compound of the invention, typically from about 5% to about 70% by weight, and more typically from about 10% to about 50% by weight, and will also depend upon the relative amounts of excipients/additives contained in the composition.

### Combinations

In specific embodiments, active agents used in combination with compounds of the present invention comprise one or more compounds generally recognized as useful for treating and/or preventing the conditions discussed herein. In one embodiment, the use of two or more drugs, which may be of different therapeutic classes, may enhance efficacy and/or reduce adverse effects associated with one or more of the drugs.

For example, in certain embodiments, the present invention provides compositions for treating and/or preventing unwanted cellular proliferation in the eye, comprising a combination of an MDM2 inhibitor and one or more anti-VEGF drugs. Such drugs include, but are not limited to, Ranibizumab (Lucentis®), Bevacizumab (Avastin®), and Pegaptanib (Macugen®). In some embodiments, the anti-VEGF drug is a protein, e.g., VEGF-Trap-Eye, a fusion protein shown to bind VEGF-A and Placental Growth Factor (PLGF). In some embodiments, the anti-VEGF drug is an antibody.

In some embodiments, the present invention provides compositions for treating and/or preventing unwanted cellular proliferation in the eye, comprising a combination of an MDM2 inhibitor and a sphingosine-1-phosphate (S1P) inhibitor (e.g., a monoclonal anti-S1P antibody, such as iSONEP™). In some embodiments, the present invention provides compositions for treating and/or preventing unwanted cellular proliferation in the eye, comprising an MDM2 inhibitor in combination with a steroid agent.

The above compounds and classes of compounds are only examples of the types of active agents that can be used in combination with an MDM2 inhibitor for the treatment and/or prevention of ocular conditions comprising unwanted cellular proliferation, and are not intended to be limiting of the invention. Rather, various further active agents can be combined with one or more compounds of the present invention according to the invention. For example, any drug generally recognized as being able to inhibit cellular proliferation can be used in combination with one or more MDM2 inhibitors according to the present invention. In some specific embodiments, any compound that has shown efficacy in treating and/or preventing any type of cancer may be combined with an MDM2 inhibitor according to the present invention. In some preferred embodiments, the present invention provides compositions free of histone deacetylase inhibitors (HDACs). Moreover, it is possible according to the invention to combine two or more additional active agents with an MDM2 inhibitor for the treatment and/or prevention of the noted conditions.

The MDM2 inhibitors discussed herein and the optional one or more other therapeutic agents may be contained within a single composition or alternatively may be administered concurrently or sequentially (consecutively) in any order. For sequential administration, each of the MDM2 inhibitor and the one or more other therapeutic agents can be formulated in its own pharmaceutical composition, each of which is to be administered sequentially, in any order. Alternatively, the compound of the formulas disclosed herein and the one or more other therapeutic agents can be formulated together. The compositions may be formulated for oral, systemic, topical, parenteral, intravaginal, intraocular, intravitreal, subconjunctival, periocular, transbuccal, transmucosal, or transdermal administration. Compositions may be designed for direct delivery to the eye, including, but not limited to, by intraocular injection, by direct injection into a given compartment of the eye, such as the vitreous, the cornea, or the retina, by application of a patch on the eye, by direct application of an ointment, spray, or droppable liquid to the eye, or by intraocular implant.

In some embodiments, the present invention provides compositions for treating and/or preventing unwanted cellular proliferation in the eye, comprising an MDM2 inhibitor, wherein the composition is used in combination with one or more other types of treatment. In some embodiments, the present invention provides compositions for treating and/or preventing unwanted cellular proliferation in the eye, comprising a MDM2 inhibitor used in combination with photodynamic therapy. In certain embodiments, the photodynamic therapy comprises treatment with Visudyne®, which acts as a dye, followed by exposure to low intensity laser light. In some embodiments, the present invention provides compositions for treating and/or preventing unwanted cellular proliferation in the eye, comprising a MDM2 inhibitor used in combination with radiation treatment. Radiation treatment as used herein refers to any type of radiation that may be used to treat such diseases, e.g., x-ray or proton beam radiation. In some embodiments, the present invention provides compositions for treating and/or preventing unwanted cellular proliferation in the eye, comprising an MDM2 inhibitor, wherein the composition is used in combination with thermal, laser, photodynamic, or transpupillary therapy.

### Methods of Use

In a further embodiment, the present invention provides a method for preventing, treating, or delaying the progression of diseases of the eye characterized by unwanted cellular proliferation, the method comprising administering a therapeutically effective amount of at least one MDM2 inhibitor to the patient.

In particular, the present invention relates to the field of treating and/or preventing abnormal, excessive, and/or unwanted cellular proliferation in the eye in animals, particularly humans and other mammals, and associated effects of these conditions. Such proliferation may occur, e.g., in tumor cells, inflammation, and/or in fibrous tissue. In one embodiment, “cellular proliferation” as used herein refers to proliferation of blood vessels, such as in angiogenesis or neovascularization. Specific ocular diseases associated with abnormal, excessive, and/or unwanted retinal vascular proliferation that may be treated or prevented according to the present invention include, but are not limited to, age-related macular degeneration, retinopathy of prematurity, diabetic retinopathy, proliferative vitreoretinopathy, ocular melanoma, ocular lymphoma, retinal vein occlusions, sickle cell retinopathy, choroidal hemangioma, choroidal arteriosclerosis, epiretinal membrane, radiation retinopathy, posterior uveitis, pathologic myopia, and ocular cancer. Those of skill in the art would readily be aware of other conditions associated with unwanted cellular proliferation in the eye, which may also be treated according to the present invention. In particular, other conditions characterized by angiogenesis and/or neovascularization may be treated according to the present invention.

The method of treatment generally includes administering a therapeutically effective amount of a compound of a formula disclosed herein, optionally in a pharmaceutical composition including one or more pharmaceutically acceptable carriers. The therapeutically effective amount is preferably sufficient to bind to MDM2 to some extent and to cause a reduction in the ability of MDM2 to interact with p53. The therapeutically effective amount is further preferably sufficient to cause some relief to the patient in the symptoms of the condition for which the patient is being treated.

The therapeutically effective dosage amount of any specific formulation will vary somewhat from drug to drug, patient to patient, and will depend upon factors such as the condition of the patient and the route of delivery. It may further be dependent on the presence of other agonists and antagonists present in the subject's system and on the degree of binding or inhibition of binding desired. When administered conjointly with other pharmaceutically active agents, even less of the compound of the invention may be therapeutically effective. Furthermore, the therapeutically effective amount may vary depending on the specific condition to be treated. Precise amounts of active ingredient required to be administered depend on the judgment of the practitioner and are peculiar to each individual.

Possible routes of delivery include buccally, subcutaneously, transdermally, intramuscularly, intravenously, orally, or by inhalation. In certain embodiments, the route of delivery used is intraocular injection, direct injection into a given compartment of the eye, such as the vitreous, the cornea, or the retina, application of a patch on the eye, direct application of an ointment, spray, or droppable liquid to the eye, or intraocular implant.

The compounds and/or formulations of the invention can be administered once or several times a day. The daily dose can be administered either by a single dose in the form of an individual dosage unit or several smaller dosage units or by multiple administration of subdivided dosages at certain intervals. In certain embodiments, there may be an initial administration followed by repeated doses at one or more hour intervals by a subsequent injection or other administration. Alternatively, continuous intravenous infusion sufficient to maintain appropriate concentrations in the blood is contemplated.

### EXAMPLES

### Reagents and Antibodies

Nutlin-3A and 3B were graciously donated by Hoffmann-La Roche, Inc. (Nutley, N.J.) and used for all experiments except for in vivo and human retinal microvascular endothelial cell (HRMEC) experiments. Racemic Nutlin-3 (Sigma, St. Louis, Mo.), which contains a 50:50 mixture of Nutlin-3A and 3B and approximately half as potent as equal concentrations of Nutlin-3A, was used for these experiments. Primary antibodies included: mouse p53 antibody (1:100 (Western blot) and 1:50 (Immunofluorescence), Santa Cruz Biotechnology, Santa Cruz, Calif.), mouse p21 antibody (1:100, Oncogene, Cambridge, Mass.), rat beta actin (1:1,000, Sigma, St. Louis, Mo.), mouse smooth muscle actin Clone 1A4 (1:100 (wholemount and cells), Dako, Carpinteria, Calif.), goat VE-Cadherin (1:100, R&D Systems, Minneapolis, Minn.), and mouse vimentin (1:25, Dako). Secondary antibodies included: HRP anti-rabbit (1:10,000, Amersham, Piscataway, N.J.) and HRP anti-mouse (1:10,000, Amersham) for Western blot analysis and Cyt and Cy₃ anti-mouse (Jackson Immunoresearch, West Grove, Pa.) were used for immunofluorescence. TO-PRO-3 (1:1000, Molecular Probes/Invitrogen, Carlsbad, Calif.) and DAPI (1:1000, Vector Laboratories, Burlingame, Calif.) were used for nuclear staining. Griffonia simplicifolia-isolectin B4 (GS-IB4)—FITC (1:200, Vector Laboratories) and GS-IB4-Alexa644 (20 μg/mL, Molecular probes, Carlsbad, Calif.) was used for staining the retinal vasculature.

### Cell Harvest

Human umbilical vein endothelial cells (HUVECs) and human umbilical vein smooth muscle cells (HUVSMCs) were isolated from the umbilical cord veins with collagenase and were cultured in M199 medium containing 10% (vol/vol) fetal bovine serum (FBS), 20 μg/ml endothelial cell growth factor, 50 μg/ml heparin, 100 μg/ml penicillin, and 100 μg/ml streptomycin in a humidified incubator at 37° C. with air/5% CO₂. HUVEC and SMC monolayers from passages 2-4 were used in these studies. Human retinal microvascular endothelial cells (HRMECs) (Cell Systems, Kirkland, Wash.) were incubated in culture containing endothelial basal medium (EBM) (Cambrex, East Rutherford, N.J.), 10% FBS, and antibiotic/antimycotic solution (Sigma; St. Louis, Mo.).

### Cell Proliferation Assay

Cells were seeded at a concentration of 2×10⁵ for HUVECs and 2×10⁴ for HUVSMCs in serum free media on 12 well plates after detachment. The cells were allowed to adhere overnight in growth media, and then incubated with X-VIVO media (Cambrex) with or without cytokines (10 ng/ml FGF-2 and 2 μg/ml heparin or 10 ng/ml VEGF-A). For HRMEC experiments, cells were seeded at a concentration of 1×10⁵ and allowed to settle overnight in 6-cm plates coated with attachment factor (Cell Signaling, Danvers, Mass.). The next day, fresh 10% FBS and EBM medium was added. For HUVEC and HUVSMC cell experiments, an equivalent dilution of 100% DMSO, Nutlin-3A, or Nutlin-3B was added at concentrations indicated in the results the following day. HRMEC experiments were performed in a similar fashion in serum with racemic Nutlin-3 because of the limited availability of Nutlin-3A and 3B. Since racemic Nutlin-3 contains a 50:50 mixture of Nutlin-3A and 3B, a higher dose of racemic Nutlin was utilized for these experiments compared to those used for HUVEC. After incubation, HUVEC were detached with collagenase/EDTA and HUVSMC and HRMEC were detached with trypsin/EDTA. The cells were manually counted with a Hemacytometer (Hausser Scientific, Horsham, Pa.) using Trypan blue exclusion.

### Matrigel Tube Assay for In Vitro Angiogenesis

Matrigel matrix (Becton Dickinson, San Jose, Calif.) was kept on ice for 24 hours. Then, 200 ill of Matrigel were added to each well of a 24 well culture plate. After hardening the Matrigel at 37° C. for 30 minutes, gels were overlaid with 500 μl of X-VIVO medium containing 3×10⁴ HUVECs. Next, endothelial cells were stimulated with 10 ng/ml of FGF-2 and 2 μg/ml heparin and then incubated with various concentrations of DMSO, Nutlin-3A, or Nutlin-3B, in triplicate, as indicated in the results. The effect of Nutlin was inspected 24 hours under an inverted light microscope. Nine overlapping images with a 10× objective were taken of each well to perform tubule length quantification. Adobe Photoshop 7.0 (Adobe Systems Inc., San Jose, Calif.) was used to make a montage image of each well. Each montage image was then imported into LSM Image Browser v3.5 (Carl Zeiss Inc., Thornwood, N.Y.) to measure tubule length. Tubule length was then standardized to the overall pixel area examined. A masked observer assessed all measurements.

### Western Blot Analysis

HUVECs and HUVSMCs were incubated at 2×10⁶ in X-VIVO media and HRMECs in EBM with 10% FBS under various conditions as reported in the results. Western blot extracts were prepared by lysing cells attached cells in cold RIPA buffer in the presence of protease inhibitors for HUVEC and HUVSMC and passive lysis buffer (Promega, Madison, Wis.) for HRMEC. After sonication and centrifugation to remove cell debris, the protein yield was quantified using the BCA protein assay kit (Pierce Biotechnology Inc., Rockford, Ill.). Normalized cell lysates were then mixed with sample buffer containing 2-mercaptoethanol and SDS and heated for 5 minutes at 95° C. Equal amounts of protein were run on SDS-polyacrylamide gels before being transferred to PVDF membranes. Binding of the primary antibody against p53, p21, and beta actin was detected with enhanced chemiluminescence reagent (Amersham) using HRP-conjugated secondary antibody (1:10,000, Amersham).

### Immunofluorescence Studies

HUVECs were seeded onto collagen coated plastic bottom culture dishes (MatTek Corp., Ashland, Mass.) that were pre-coated with 0.2% gelatin. Nutlin-3A and 3B were diluted to a concentration of 5 μM in X-VIVO media for 8 hours. Immunofluorescence images with a direct light microscope and fluorescent light were captured using a 3CCD camera and Qcapture imaging software (Qcapture v 2.81, Quantitative Imaging Corp., Burnaby, BC Canada) or a Carl Zeiss LSM 510 confocal microscope (Carl Zeiss Inc., Thornwood, N.Y.).

### Flow Cytometric Analysis for Apoptosis

HUVECs and HUVSMCs were incubated with 7.5 μM of Nutlin-3A or DMSO for 24 hours (HUVEC) and 48 hours (HUVSMC) in X-VIVO media with FGF-2 (10 ng/mL) and heparin (2 μg/mL). HUVECs were detached with collagenase/EDTA and HUVSMCs were detached with trypsin/EDTA. The cells were then washed twice with PBS, and stained with propidium iodide and annexin V-FITC (Annexin V-FITC apoptosis detection kit I, Beckton Dickinson) according to the manufacturer's instructions.

### TUNEL Assay for Apoptosis

HUVECs were plated on collagen coated plastic bottom culture dishes (MatTek Corp., Ashland, Mass.) overnight. HUVECs were then treated with varying concentrations of Nutlin-3A and 3B in X-VIVO medium for 24-72 hours. HRMEC were treated with 15 μM of racemic Nutlin for 24 hours in 10% FBS. The TUNEL assay was performed following instructions provided with the kit (Roche Applied Science, Indianapolis, Ind.). Representative images of the HRMEC experiment were provided with a 4× objective using an Olympus AX70 fluorescence microscope following the addition of a nuclear counter stain. For HUVEC experiments, 5 representative fields with a 20× objective using a Zeiss confocal microscope were captured. 2 masked observers performed cell counts. For wholemount retina staining, TUNEL solution was incubated for 48 hours diluted in PBS plus 0.3% Triton-X 100 after lectin staining.

siRNA p53 for HUVEC

Passage 3 HUVECs were grown to confluence. pRETRO-SUPER and pRETRO-SUPER-p53 viruses were produced by GP2 cells (Clontech, Mountain View, Calif.). Supernatant was filtered and then added to HUVECs in the presence of 4 mg/ml polybrene followed by 1 μg/ml of Puromycin selection for 7 days.

### Quantitative RT-PCR for Bax-BCL2

Quantitative RT-PCR (qPCR) was performed using an ABI 7500 fast System (Applied Biosystems, Foster City, Calif.) in standard mode. The software used to analyze the data was SDS v. 1.3.1 (Applied Biosystems). qPCR was performed according to ABI standard protocols. The following primer/probe sets were obtained through Applied Biosystems (sense and anti-sense): beta-actin, BAX, and BCL-2. The delta Ct method was used to obtain relative quantification, i.e. threshold cycle (Ct) values of the target gene (BAX, BCL-2) were normalized to the corresponding Ct value of the control gene (beta-actin). Relative expression was calculated as follows: relative expression=(2^{-ΔCt})*10000. NTC and minus RT controls were run appropriately.

### Subcutaneous and Periocular Injection for In Vivo Retinal Development Assay

A technique described by Strombland et al. was modified to study the effects of Nutlin-3 on in vivo vascular development (see Stromblad S, Fotedar A, Brickner H, et al., J. Biol. Chem. 277:13371-13374 (2002), which is incorporated herein by reference. Briefly, racemic Nutlin was administered by subcutaneous injection in the nape of the neck or in the periocular area of each eye to wild type 129 S1-VIMJ (Jackson Laboratories, Bar Harbor, Me.) mouse pups within 12 hours of birth. The mice received a total of 4 (periocular experiments) or 5 (subcutaneous neck experiments) injections of either racemic Nutlin or 100% DMSO. The first injection of Nutlin was administered at a dose of 40 mg/kg while the rest were given at a dose of 80 mg/kg for experiments involving injections in the nape of the neck. A dose of 80 mg/kg was administered for all injections in the periocular series of experiments. The pups were euthanized on postnatal day 3 and the eyes were enucleated after the fused eyelids incised.

### Intravitreal Injection for In Vivo Adult Retinal Vessel Assay

Adult, three month old, 129 and C57/BL6/129S mice were anesthetized with Avertin and also given a drop of topical proparacaine 1% for local anesthesia. Using a stereo microscope, a glass capillary pipette was used to inject 1 μL of either vehicle or racemic Nutlin-3 into the vitreous of cavity of both eyes of each animal. Mice were then euthanized five days after injection. All procedures involving mice were approved and monitored by the Weill Medical College of Cornell University and Hospital for Special Surgery Animal Care and Use Committees.

### Preparation of Retinal Wholemounts

The eyes were then fixed in 4% paraformaldeheyde overnight and washed three times with PBS. After removing the cornea and lens, the hyaloidal (primitive) vasculature was removed and four radial incisions were made in the eyecup to flatten the retina/choroid/sclera complex. The choroid and sclera were removed from the retina and cut at the optic nerve. After the retinal wholemount was blocked with 5% bovine serum albumin, 5% normal donkey serum, and 0.5% TritonX-100 for 3 hours or overnight, the wholemount was incubated with a 1:200 dilution of GS-IB4 lectin overnight and mounted with Vectashield (Vector Laboratories). For double staining wholemount experiments, retinas were incubated with primary antibody overnight at 4° C., underwent 6 one hour washes with PBS-T, followed by incubation with secondary antibody overnight at 4° C., followed by 6 one hour washes with PBS-T prior to being cover-slipped with Vectashield. Using a fluorescent biomicroscope (Carl Zeiss Discovery V12, Thornwood, N.Y.) hyaloidal vessels, vessels connected to the optic nerve, on the surface of the retina, were dissected in a masked manner. A confocal laser (Carl Zeiss LSM 510 meta) was used to obtain images of the retinal vasculature.

### Quantification of Retinal Vascular Development Model

We captured images with the same laser power, objective, gain, pinhole, and amplifier offset. The resultant images were masked to the analyst assessing the images. All analyzed images were captured with a 5× objective and then exported to Adobe Photoshop 7.0 (San Jose, Calif.) as high-resolution .tiff images. Using the Magic Wand tool and histogram function, the analyst recorded the number of pixels that best represented the retinal vasculature and omitted the residual hyaloidal (fetal) vasculature that was unable to be removed during dissection. Blood vessels assessed to be residual hyaloid were found at a different focal plane than retinal blood vessels and were usually connected to the optic nerve.

### Statistical Analysis

Comparison values were expressed as a percentage or fold difference (means±SEM). P-values <0.05 were considered significant and calculated using the Student's t test in Microsoft Excel (Microsoft Corp., Redmond, Wash.).

### Results

### Nutlin-3 Inhibits Endothelial Cell and Smooth Muscle Proliferation

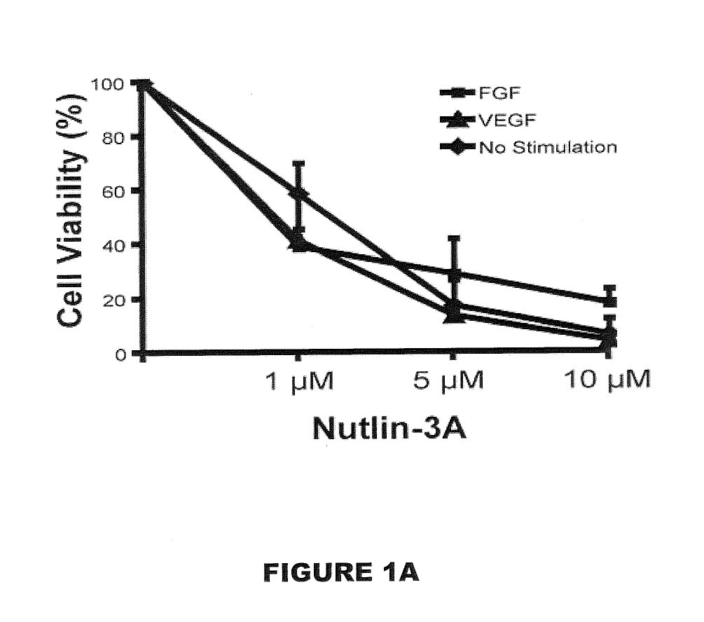

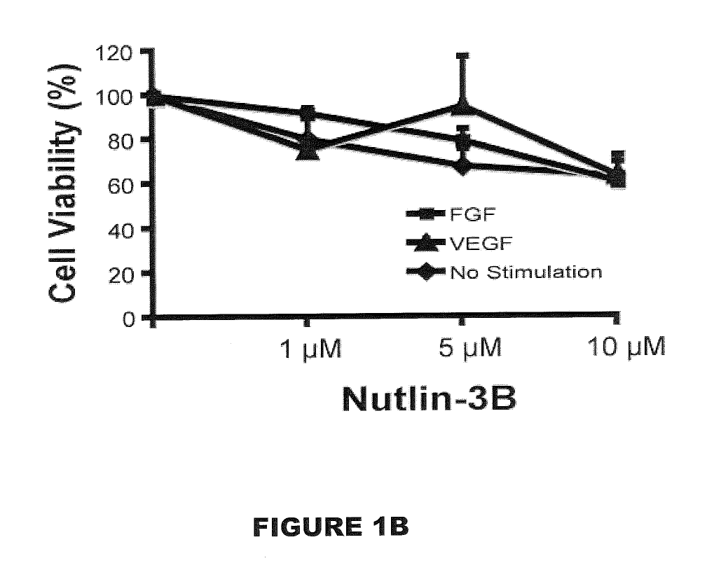

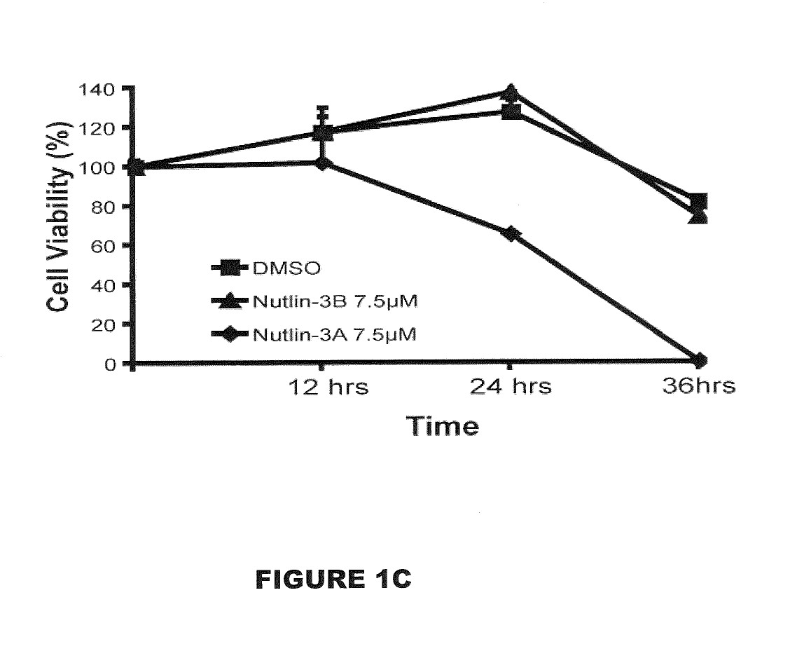

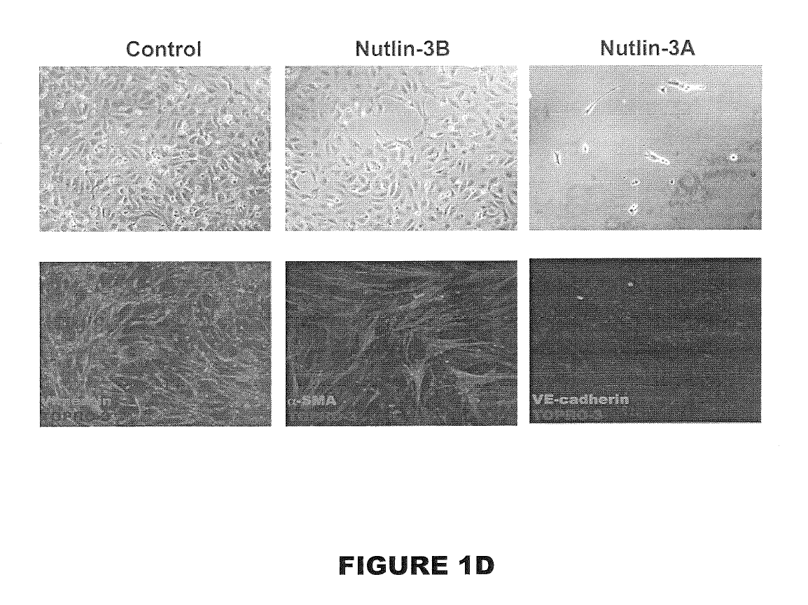

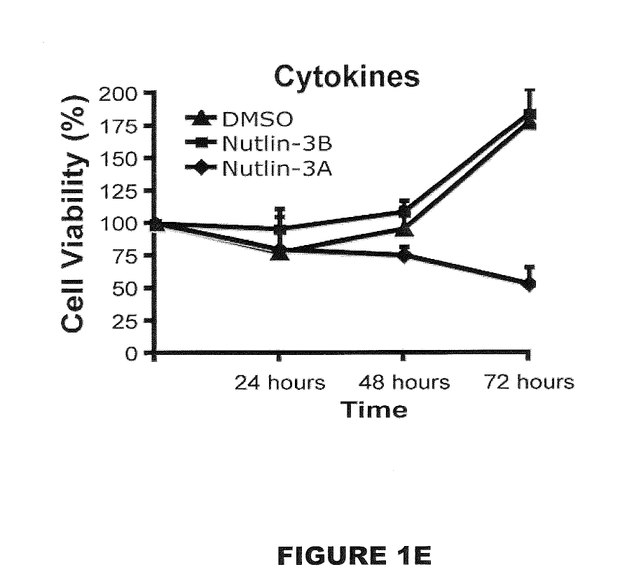

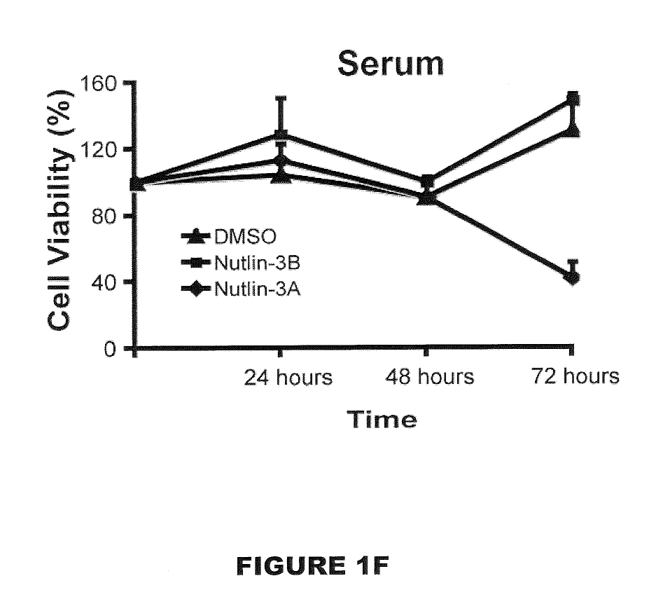

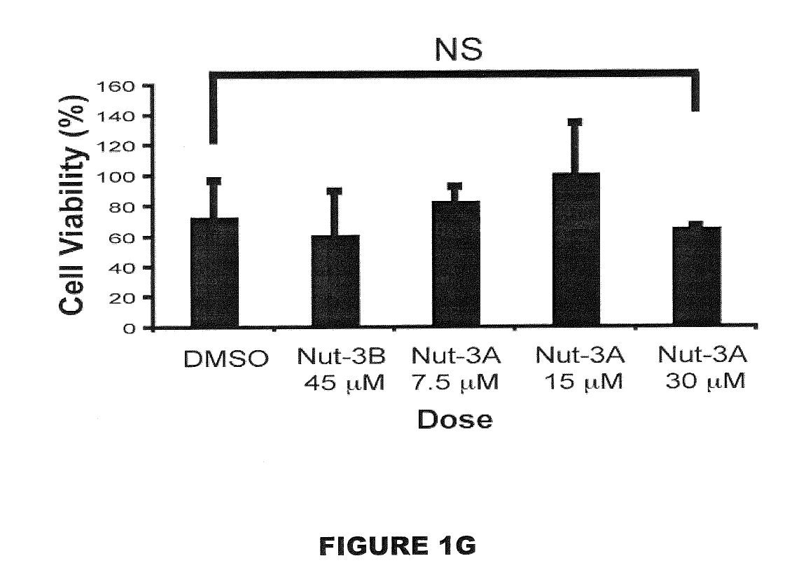

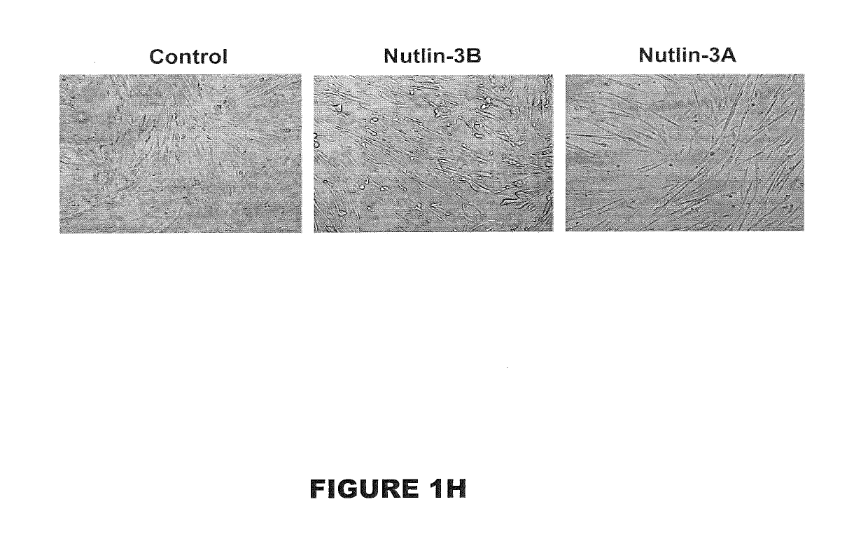

We hypothesized that Nutlin-3A is able to inhibit endothelial cell proliferation. First, we performed a dose response curve on unstimulated and stimulated serum free HUVECs. FIG. 1A is a series of graphs relating to Nutlin-3A or Nutlin-3B, in 1, 5, or 10 μM concentrations, which was added to proliferating HUVECs for 36 hours (upper left and right panels). HUVECs were either unchallenged or challenged with VEGF-A or FGF-2 during the incubation. Vehicle (DMSO), Nutlin-3A 7.5 μM or Nutlin-3B 7.5 μM were added to 2×10⁵ serum free, unchallenged HUVECs (lower panel). Cell viability was measured at 12, 24, and 36 hours after incubation.

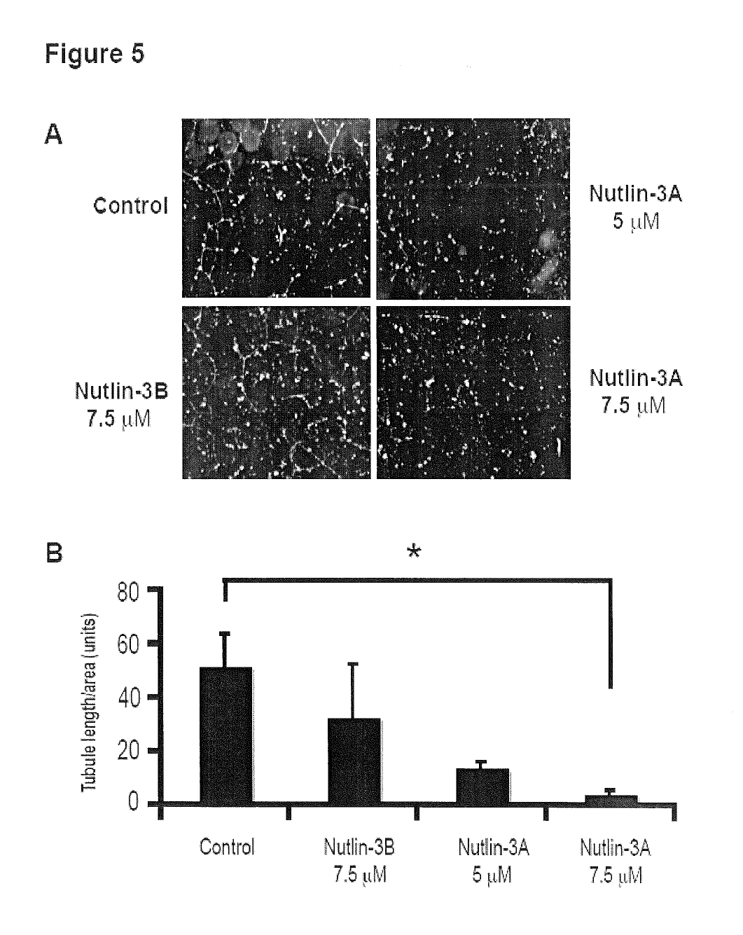

We found that Nutlin-3A is able to inhibit HUVEC proliferation at all tested doses with greater activity at higher concentrations in both conditions (FIG. 1A). Similar experiments were conducted with 10 μM Nutlin-3B, an inactive enantiomer of Nutlin-3A, which had little effect on HUVEC proliferation (FIG. 1B). A time course experiment performed using 7.5 μM of Nutlin-3A demonstrates inhibition of unstimulated, serum free HUVEC growth with near complete cell death by 36 hours (FIGS. 1C and 1D, top row). Since smooth muscle cells play an important role in angiogenesis, we investigated the possibility that Nutlin-3 may inhibit HUVSMCs. We characterized our cells with antibody staining, and found that these cells are smooth muscle actin and vimentin positive, but VE-cadherin negative consistent with vascular smooth muscle cells (FIG. 1D, bottom row). We next performed, time course experiments, using 7.5 μM of Nutlin-3A, which revealed that proliferating serum free HUVSMCs, either cytokine activated with FGF-2 (FIG. 1E) or 5% FBS (FIG. 1F), are growth inhibited at 48 to 72 hours (FIG. 1H). Taken together with the HUVEC experiments, this data suggests that Nutlin-3A mediated effects take longer in HUVSMCs compared to HUVECs. To confirm this is not a dose related issue, we added increasing concentrations of Nutlin-3A to serum free, unchallenged HUVSMCs (0, 7.5, 15, 30 μM), 45 μM of Nutlin-3B served as a toxicity control, and did not find an early response even with higher concentrations of Nutlin-3 at 36 hours (FIG. 1G) (p>0.05).

### Nutlin-3 Induces p53 Expression and Downstream Targets in HUVEC and HUVSMC

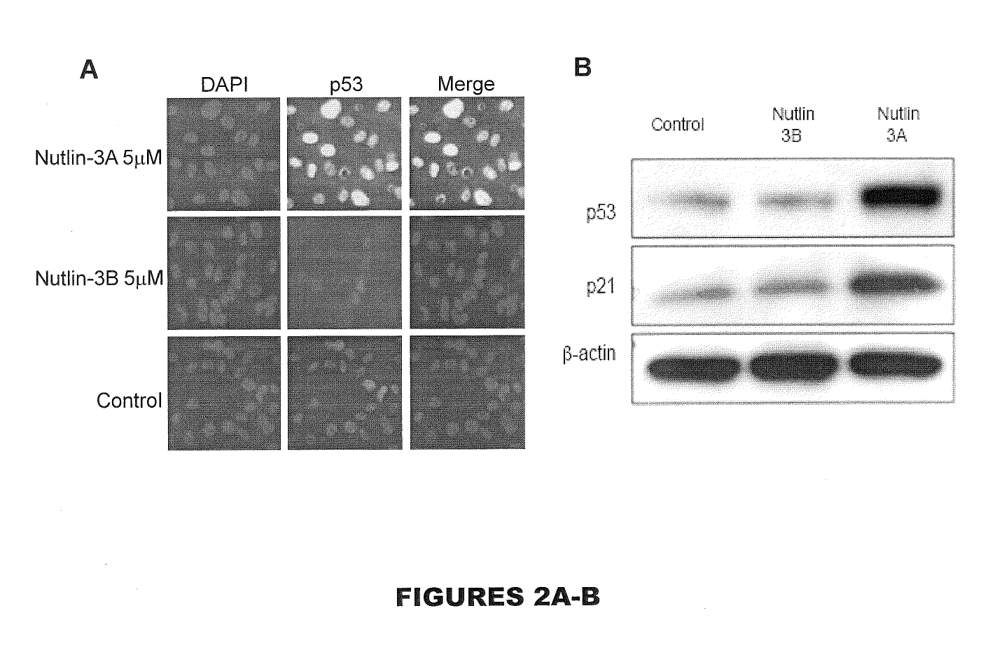

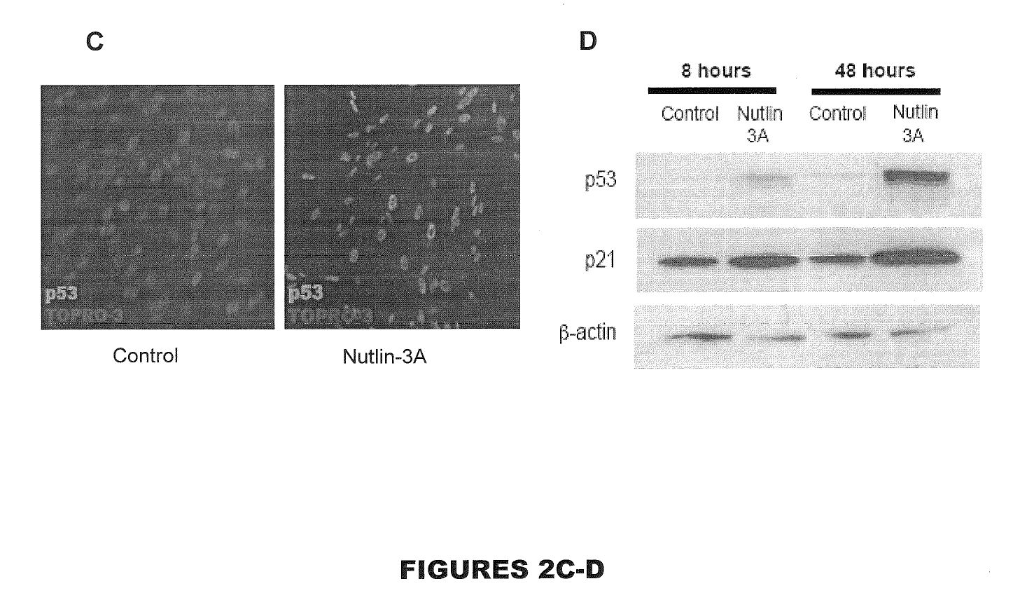

Next, we investigated the mechanism that Nutlin-3 inhibits HUVEC and HUVSMC proliferation. First, we examined if Nutlin-3A induces a p53 response in HUVEC. We performed immunofluorescence and Western blot studies for p53 and downstream targets. After 8 hours of incubation with Nutlin-3A, we observed increased nuclear expression of p53 on immunofluorescence staining compared to control (FIG. 2A). In FIG. 2A, hyperintense cells observed in the top row of the middle column demonstrate p53 expression in the cells. Lack of hyperintense cells in the remaining panels of the middle column indicate that there was little, if any, p53 expression in the cells with these conditions. DAPI is a reference marker that labels the nucleus of the cells that were examined. Western blot revealed increased p53 protein, and its downstream target p21, compared to Nutlin-3B or control (FIG. 2B). Subsequently, we examined the possibility that HUVSMC had a delayed response to Nutlin-3 secondary to an attenuated p53 response. Interestingly, we found that at early time points and a relatively low dose, within 8 hours of 7.5 μM respectively, Nutlin-3A initiated a p53 response in the HUVSMC both on immunofluorescence (FIG. 2C) and Western blot (FIG. 2D) studies. This suggests that Nutlin-3A initiates a p53 response in HUVSMC at an equivalent time and dose similar to HUVEC.

### Nutlin-3 Induces Apoptosis in HUVEC but not in HUVSMC

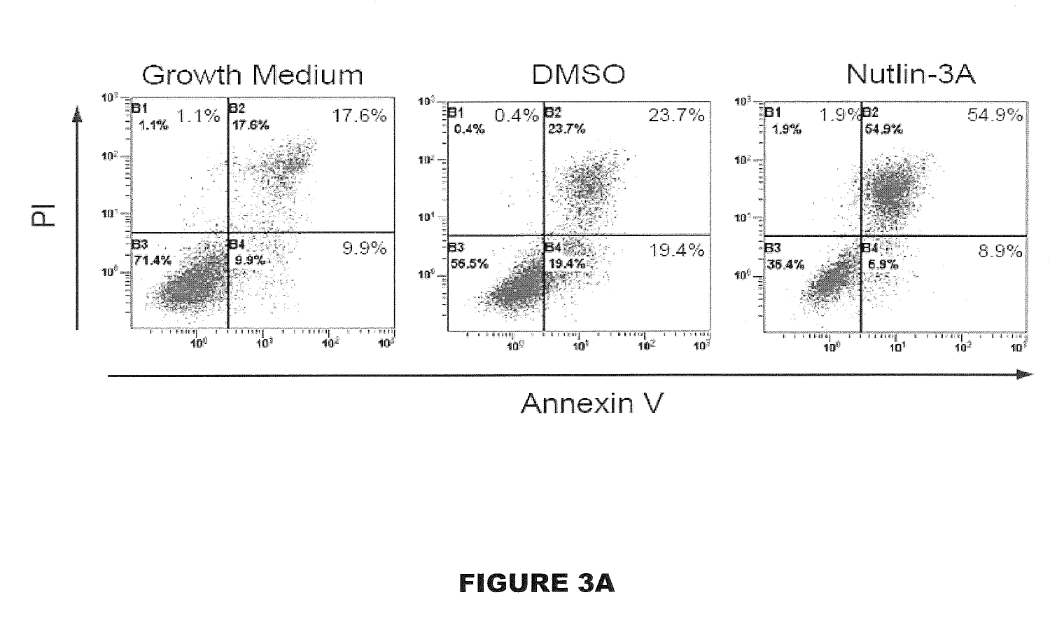

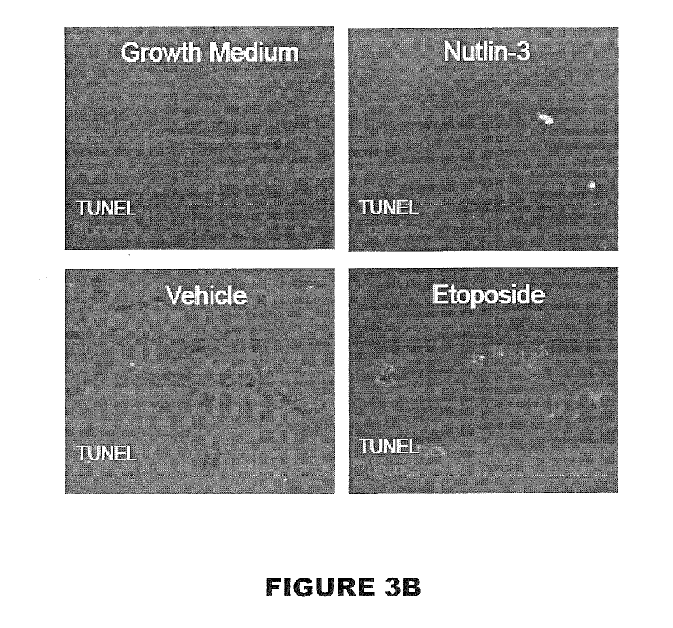

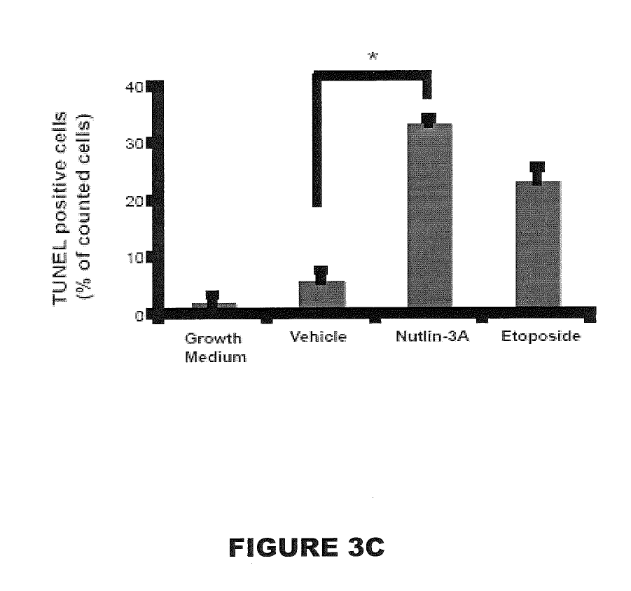

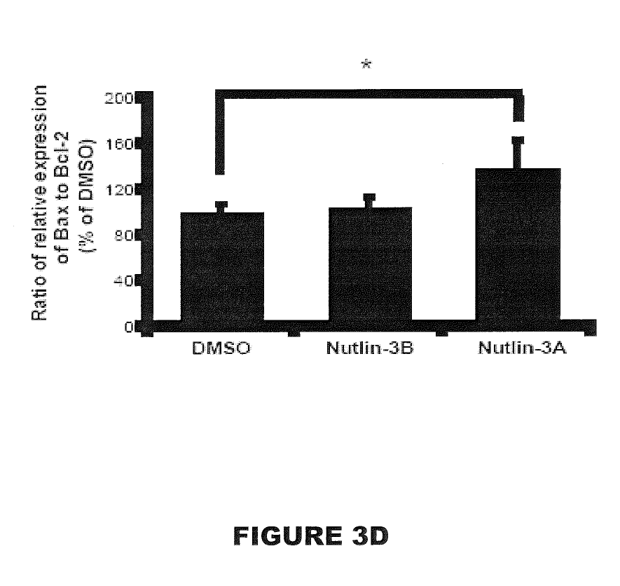

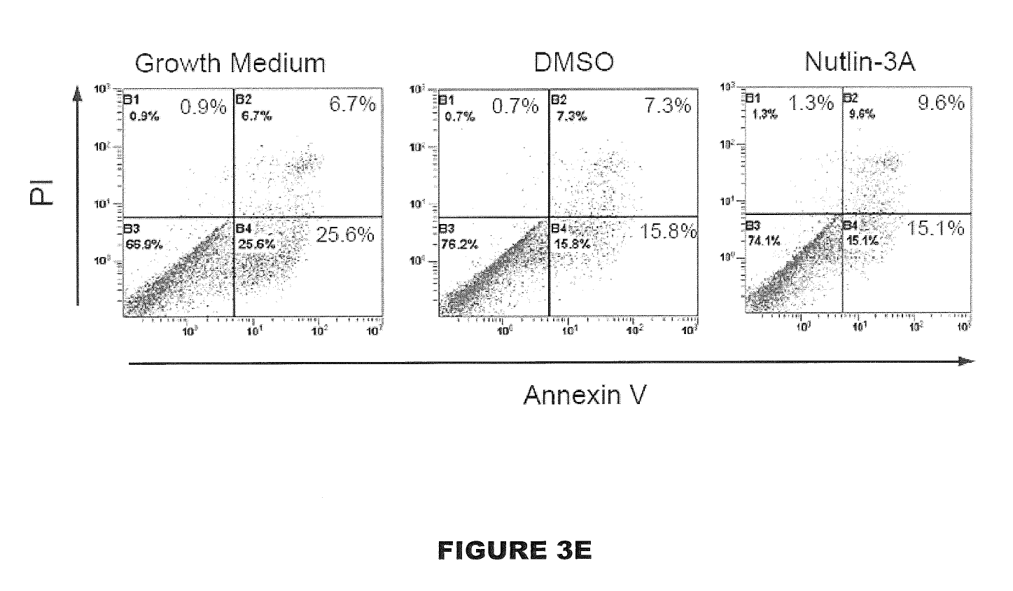

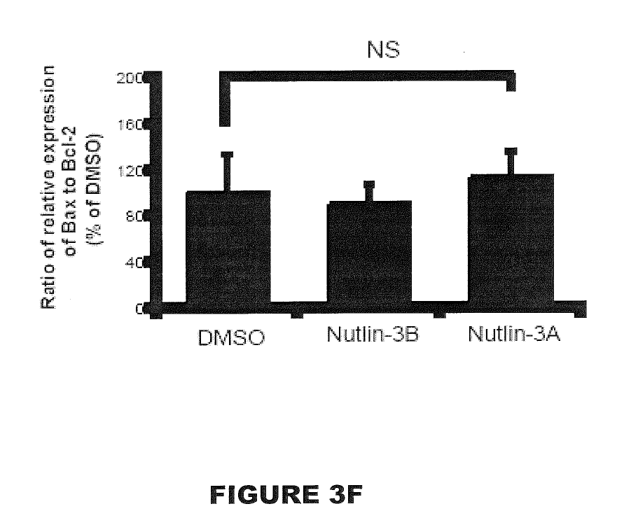

Next, we wanted to study the mechanism of Nutlin-3A inhibited cell viability in HUVEC cultures. We predicted that activation of the p53 pathway and subsequent downstream targets may initiate the apoptosis pathway. We used flow cytometry to analyze results of annexin V and propidium iodide staining for markers of early and late apoptosis. After 24 hours of incubation with Nutlin-3A, we found significantly more double positive annexin V and propidium iodide cells, a marker of late apoptosis, compared to the control (FIG. 3A). Also, we performed quantitative RT-PCR to look for relative expression of BAX, a marker of apoptosis, and BCL-2, an anti-apoptosis gene, in Nutlin-3A treated HUVECs. We found a relative increase in the ratio of BAX/BCL-2 in Nutin-3 treated HUVECs suggesting that these cells are undergoing apoptosis (FIG. 3D). Next, we performed the TUNEL assay as another method to detect apoptosis. TUNEL assay demonstrated that there were significantly more cells undergoing apoptosis compared to control in serum free HUVEC (FIG. 3B,C). Taken together, these experiments suggest that Nutlin-3 activates the apoptosis pathway in serum free HUVEC. Based on the observation that Nutlin-3A activates the p53 pathway, we explored the possibility that smooth muscle cells were also undergoing apoptosis. We analyzed apoptosis at 36 hours in HUVSMC because we determined from our earlier work that proliferation is inhibited at a later time point compared to HUVEC. Unexpectedly, we found there was no significant difference between the Nutlin-3A treated HUVSMCs compared to control in the annexin V and propidium iodide experiments (FIG. 3E). We also performed quantitative RT-PCR on HUVSMCs and looked for expression of BAX and BCL-2 genes. There was not a significant increase in the BAX/BCL-2 ratio suggesting that these cells are not undergoing apoptosis (FIG. 3F).

### p53 is Necessary for Nutlin-3 Mediated Cell Death

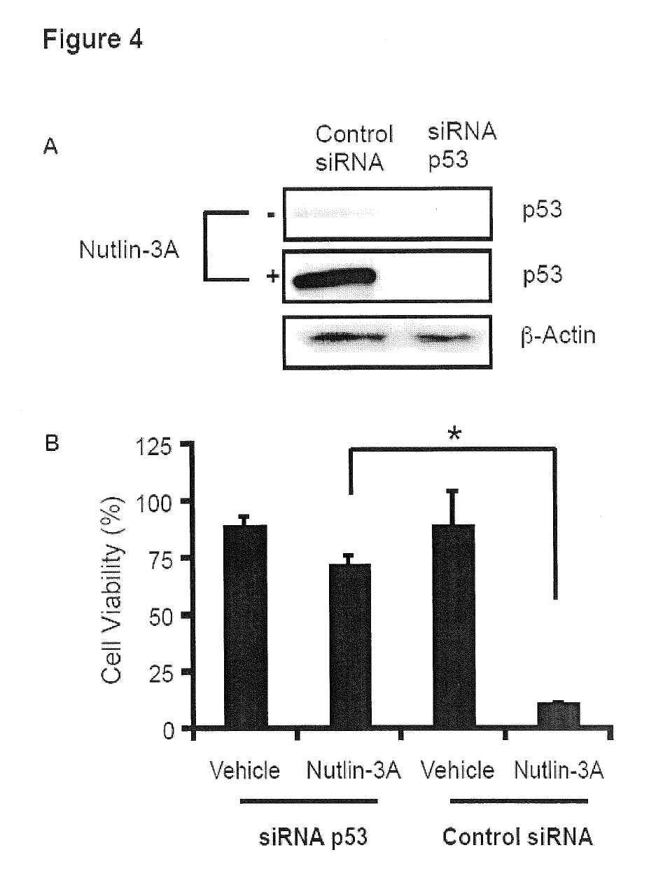

To determine if the p53 pathway is necessary for the observed Nutlin-3 mediated cellular effects, we infected HUVEC with retrovirus expressing either control short interfering RNA (siRNA) or p53 siRNA. FIG. 4 demonstrates that p53 is necessary for Nutlin-3 mediated cell death. (A and B) HUVECs were infected with a lentiviral vector expressing p53 siRNA or control siRNA for 48 hours and then underwent Puromycin selection for 72 hours. We determined the efficacy of our siRNA silencing by probing for p53 protein on Western blot on Nutlin-3A treated HUVECs which showed almost complete suppression of p53 protein (FIG. 4A). We also performed a cell viability assay, and found that p53 siRNA infected cells had become resistant to Nutlin-3A (FIG. 4B). This data suggests that the p53 pathway is necessary for Nutlin-3A mediated inhibition of HUVEC cell viability.

### Nutlin-3 Inhibits Capillary Tube Formation

To better understand if this mechanism for HUVEC death would be applicable to angiogenesis, we performed a capillary tube formation assay that measures the ability to form tube like structures. Our experiments revealed a 93% reduction in capillary tube formation between Nutlin-3A treated HUVEC compared to control (FIG. 5A,B). *p<0.005. Also, the effects of Nutlin-3A on inhibiting capillary tube formation appear to be dose dependent (FIG. 5B).

### Nutlin-3 Inhibits Retinal Vascular Development

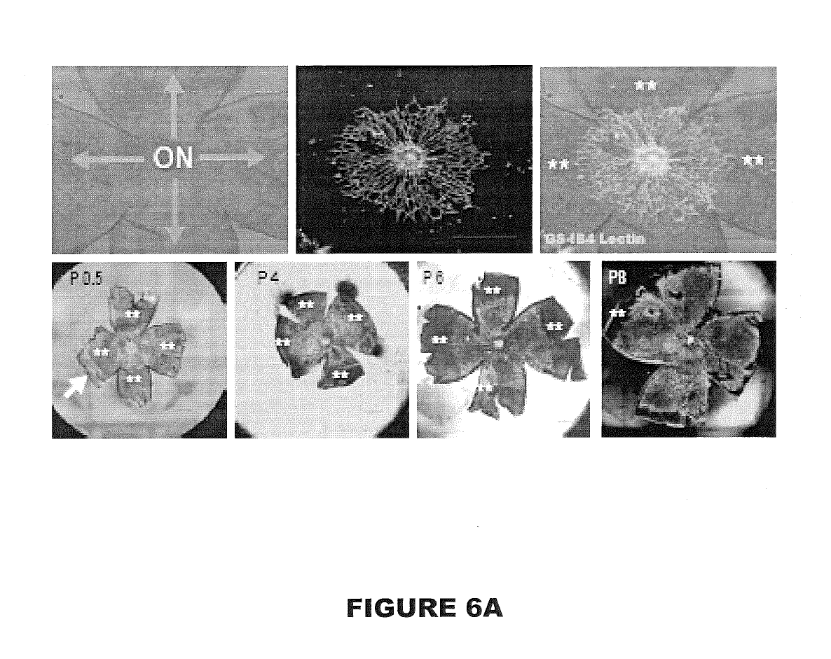

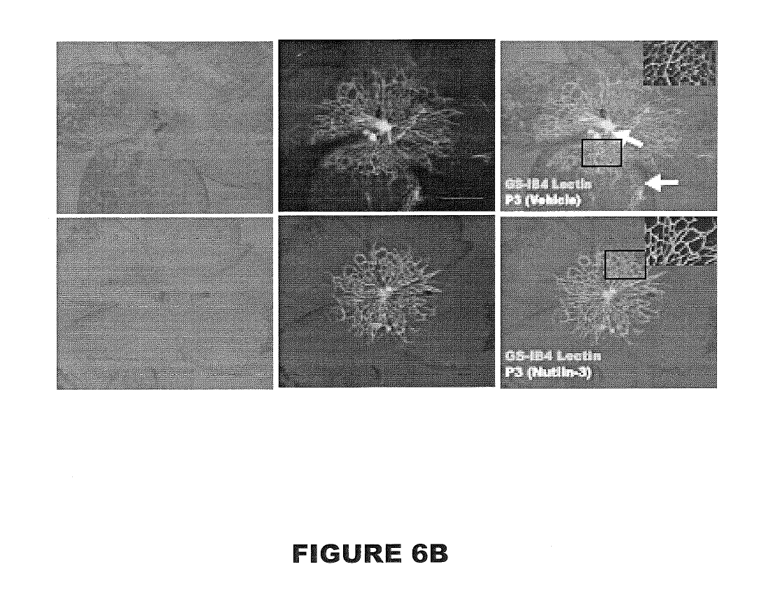

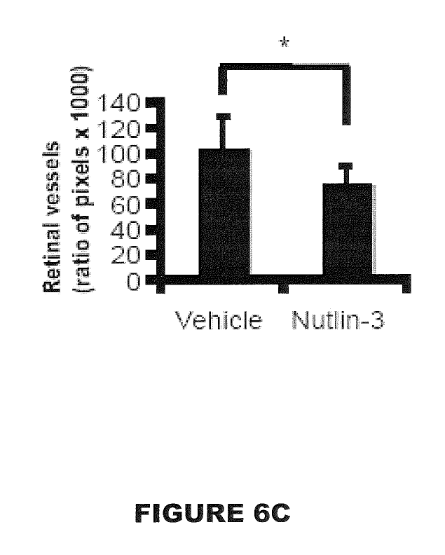

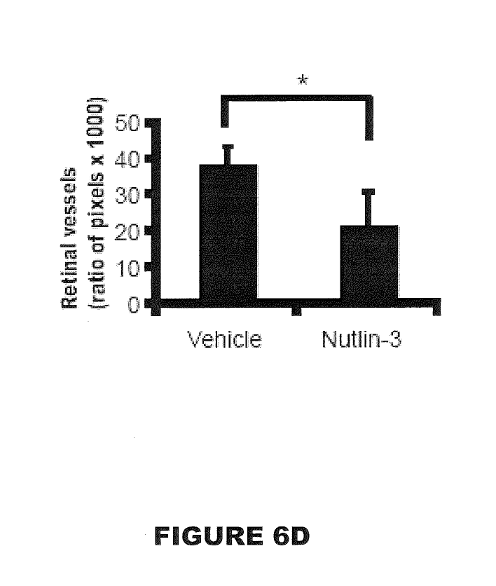

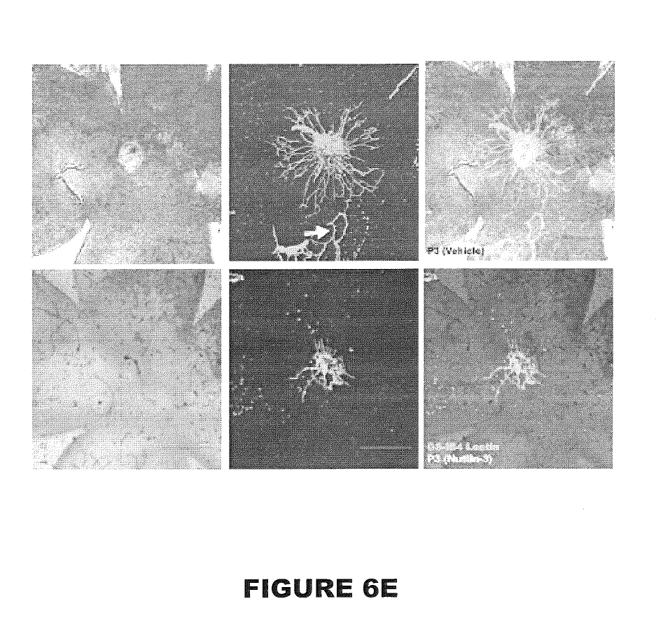

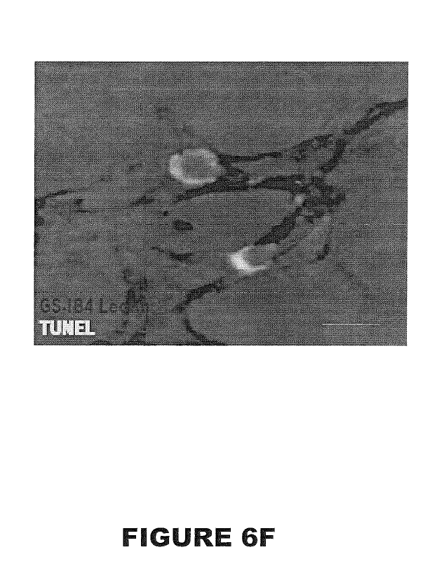

Next, we used a retinal development model to test the possibility that Nutlin-3 could be used to inhibit retinal vessel proliferation (FIG. 6A). FIG. 6A illustrates postnatal mouse retinal vascular development after birth (upper left panel). The illustration depicts the optic nerve (ON) at the center of a retinal wholemount with green arrows indicating the direction of postnatal blood vessel growth (upper middle and right panels). GS-IB4 lectin staining of a retinal wholemount with double asterisks (**) indicates areas of avascular retina in the developing mouse pup (lower panels). Images from left to right show radial growth pattern of post-natal development of retinal vasculature. White arrow indicates remaining residual fetal vasculature after dissection. (B and C) Neonatal mice were given subcutaneous injections in the nape of the neck. (B) The retinal vasculature is abrogated in the Nutlin-3 treated eyes (n=6) (bottom row) compared to the sham injected mice (n=4) (middle row). In addition, we noticed loss of smaller caliber vessels (inset, right column) in the Nutlin-3 treated mice. White arrows point to the residual hyaloidal (fetal) vasculature that could not be removed during dissection. We found that subcutaneous Nutlin-3 injection in the nape of the neck revealed a modest (27.4%), but statistically significant reduction (p<0.05), in the amount of retinal vessels compared to sham injected mice (FIG. 6B,C). In an attempt to reduce systemic toxicity and improve delivery to the eye, we performed similar experiments and injected Nutlin-3 in the periorbital area under the fused eyelid. In these experiments, we found 43.8% fewer blood vessels compared to the sham-injected mice (p<0.01) (FIG. 6D,E). Although a rare event, we were able to detect TUNEL positive cells that co-localized to the retinal endothelium (Griffonia simplicifolia-isolectin B4 (GS-IB4) lectin stained) in a Nutlin-3 treated eye (FIG. 6F).

Nutlin-3 does not Target Pre-Existing Blood Vessels

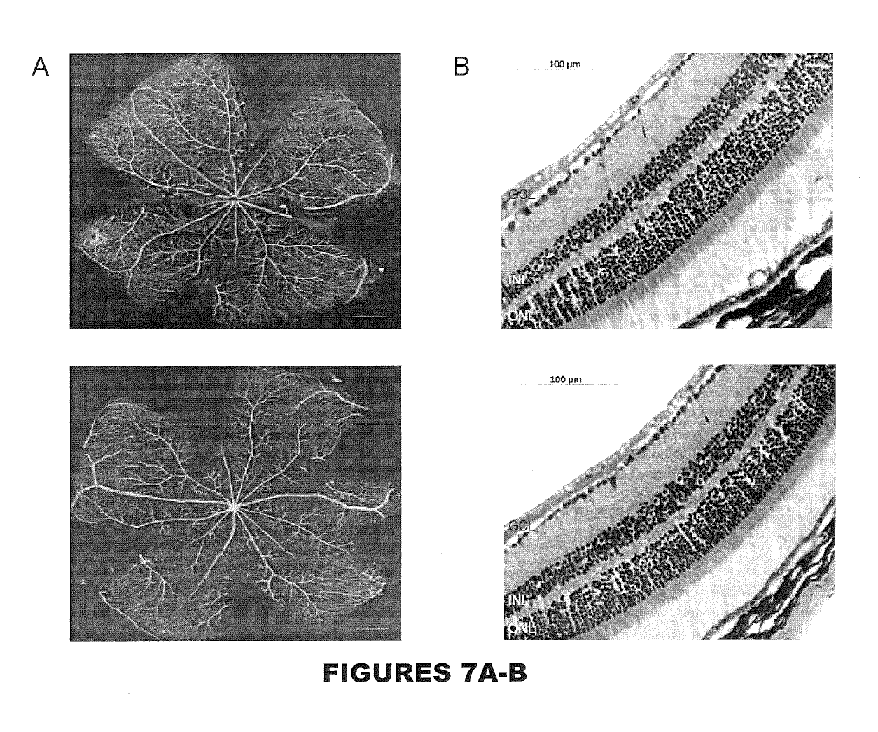

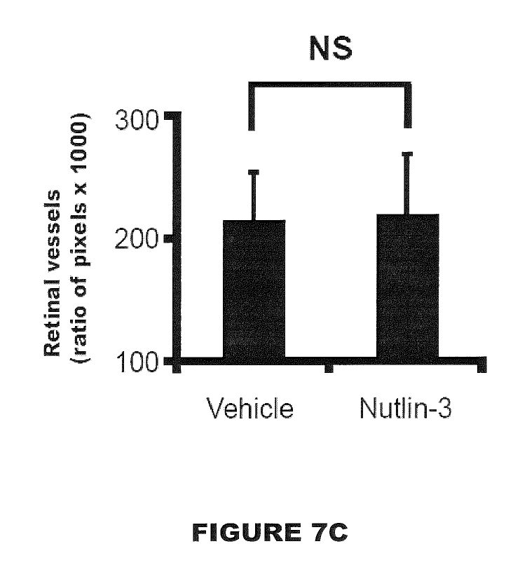

Thus far we examined the effect of Nutlin-3A on proliferating cells in vitro and in vivo. To test whether Nutlin-3A has an effect on established, non-proliferating blood vessels, we injected Nutlin-3A in the vitreous cavity of adult mice. Our analysis demonstrated that there was no reduction of the retinal vasculature in the Nutlin-3 treated eyes compared to the control (p>0.05) (FIG. 7A,C). In addition, we were interested in examining the architecture of the neurosensory retina to study possible effects on neuronal cells. On hematoxylin and eosin staining, we did not observe any gross changes in the thickness of the neuronal cell layers of the Nutlin-3 treated eyes (FIG. 7B). These data suggest that Nutlin-3 does not cause vascular obliteration of existing retinal vessels or is grossly toxic to the neurosensory retina.

### Discussion

The data disclosed herein suggests that radiation is not a prerequisite for endothelial cell apoptosis. While we confirmed the finding that Nutlin-3 treated endothelial cells do not undergo apoptosis in serum enriched media, our experiments revealed that they will undergo apoptosis in serum free defined media without requiring additional compounds such as a PI3 kinase inhibitor.

In addition to the HUVEC data, we performed experiments in human retinal microvascular endothelial cells (HRMECs) to explore differences between macrovascular and microvascular endothelial cells. Similar to HUVECs, loss of MDM2 by Nutlin-3 caused an upregulation of p53 and was sufficient to inhibit cell viability. One notable difference between HUVECs and HRMECs is that Nutlin-3 induces apoptosis in HRMECs in serum while HUVECs do not. This data suggests that in serum, HRMECs are more likely to undergo apoptosis in response to Nutlin-3 mediated MDM2 inhibition than HUVECs.

We chose a murine retinal vascular development model to further interrogate the role of Nutlin-3 as a solitary agent for inhibiting in vivo angiogenesis. Normal murine retinal vascular development begins in utero and continues after birth (FIG. 6A). This model is advantageous for the following reasons: 1) the system does not rely on a tumor model so we could study the direct effect of MDM2 inhibition on blood vessels, and 2) this in vivo model does not require the addition of supra-physiological doses of pro-angiogenic cytokines.

Our in vivo data demonstrate that Nutlin-3 is capable of inhibiting retinal vascular development. We were able to show this using two slightly different models of drug delivery. In our systemic subcutaneous injection experiments, we demonstrated a modest, but statistically significant, inhibition of the retinal vasculature between Nutlin-3 treated mice and sham-injected mice. This difference was accentuated when we delivered periocular injections presumably due to higher concentrations of the drug at the local site. Periocular drug delivery was chosen because it is an established route of administration commonly used in clinical practice. In addition, this method of drug delivery does not cause retinal trauma; an important factor for quantitative analysis in neonatal mouse pups with small retinas. Also of note, experiments evaluating either route of drug delivery were performed with controls within the same litter. Several independent experiments were performed, and the largest litters were represented. We discovered variations in retinal vascular growth between litters of the same aged mice precluded the ability to analyze all the mice as one group. This may be due to differences in litter size, where larger litters produced on average smaller pups and smaller litters produced relatively larger pups. We hypothesize, like humans, smaller pups have differences in retinal vascular maturation compared to larger pups of the same age. Therefore, intra-litter analysis was only performed.

In addition to its effects on endothelial cells, our study is the first to show that Nutlin-3 inhibits the in vitro proliferation of smooth muscles cells (SMCs) (FIGS. 1E-1G). We discovered that HUVSMCs initiate a rapid p53 response to Nutlin-3 similar to HUVEC, but HUVSMCs may not be as sensitive to p53 up-regulation witnessed by a delay in cell death. Interestingly, we did not find that Nutlin-3 treated HUVSMCs undergo apoptosis like HUVEC. In our mouse retina model, we found that first and second order vessels can have smooth muscle cells around blood vessels, suggested by smooth muscle actin wholemount antibody staining. In our analysis, we observed that Nutlin-3 preferentially targeted capillaries and smaller blood vessels. This suggests the possibility that blood vessels ensheathed by smooth muscle cells may be protected from Nutlin-3.

We studied the possibility that Nutlin-3 could have unintended effects on established, non-proliferating retinal blood vessels. Additionally, we examined the possibility that Nutlin-3 could have an effect on neuronal cells in the neurosensory retina since these cells also have wild-type p53. Our analysis did not identify gross changes in neuronal thickness of the three major neuronal layers in the retina (FIG. 7B). However, this does not exclude the possibility that there is vehicle related toxicity or subtle changes in neuronal density or function. In addition, we did not see a difference quantitatively or qualitatively between Nutlin-3 and sham-injected adult mouse retina, suggesting that Nutlin-3 has minimal or no effect on established blood vessels. Taken together, this suggests that Nutlin-3 preferentially targets developing retinal vessels and has no effect on pre-existing blood vessels. A limitation of these experiments is that we performed an intravitreal injection instead of repeated periocular injections. We were required to change the delivery method since adult mice do not have fused eyelids.

In summary, our results demonstrate that Nutlin-3 can inhibit human smooth muscle and endothelial cell proliferation, and that a functional p53 pathway is necessary for Nutlin-3 mediated effects on both microvascular and macrovascular endothelial cells. Nutlin-3 leads to accumulation of p53 resulting in apoptosis in HUVECs and HRMECs. This work suggests that retinal vascular development is sensitive to MDM2 inhibition through the p53 pathway in mice.

That which is claimed:

1. A method of treating an eye disease associated with unwanted cellular proliferation in the eye of a subject in need thereof, the method comprising administering to the subject an effective amount of an MDM2 inhibitor.

2. The method according to claim 1, wherein the MDM2 inhibitor is a nutlin compound.

3. The method according to claim 2, wherein the nutlin compound is Nutlin-3.

4. The method according to claim 1, wherein the subject has a condition selected from the group consisting of age-related macular degeneration, retinopathy of prematurity, diabetic retinopathy, proliferative vitreoretinopathy, ocular melanoma, ocular lymphoma, retinal vein occlusions, sickle cell retinopathy, choroidal hemangioma, choroidal arteriosclerosis, epiretinal membrane, radiation retinopathy, posterior uveitis, pathologic myopia, and ocular cancer.

5. The method according to claim 1, wherein the subject is a human.

6. The method according to claim 1, wherein the MDM2 inhibitor is delivered intraocularly.

7. The method according to claim 6, wherein the MDM2 inhibitor is delivered intravitreally.

8. A pharmaceutical composition comprising one or more MDM2 inhibitors and one or more ophthalmologically acceptable excipients, the composition being adapted for intraocular delivery.

9. The pharmaceutical composition according to claim 8, wherein the MDM2 inhibitor is a nutlin compound.

10. The pharmaceutical composition according to claim 9, wherein the nutlin compound is Nutlin-3.

11. The pharmaceutical composition according to claim 8, wherein the composition is formulated for intravitreal delivery.

12. The pharmaceutical composition according to claim 8, further comprising another compound recognized as effective in the inhibition of cellular proliferation.

13. The pharmaceutical composition according to claim 12, wherein the compound comprises an anti-VEGF drug.
